(19) United States
(12) Patent Application Publication
(10) Pub. No.: US 2016/0312261 A1
(43) Pub. Date: Oct. 27, 2016

(54) ENZYMATIC PRODUCTION OF CYTOSINIC NUCLEOSIDE ANALOGUES

(71) Applicant: PLASMIA BIOTECH, S.L., Badalona (ES)

(72) Inventors: Marta PASCUAL GILABERT, Mollet del Valles (ES); Victor M. Deroncelé Thomas, Mollet del Valles (ES); Rafael Montilla Arévalo, Mollet del Valles (ES)

(21) Appl. No.: 14/696,071

(22) Filed: Apr. 24, 2015

(51) Int. Cl. C12P 19/38

(52) U.S. Cl. CPC C12P 19/385

(57) ABSTRACT

The invention relates to the enzymatic production of cytosinic nucleoside analogues. In particular it relates to a new synthesis process of cytosine nucleoside analogues by using nucleoside phosphorylase enzymes, particularly Pyrimidin Nucleoside Phosphorylases (PyNPs) or mixtures of Purine Nucleoside Phosphorylases (PNPs) and PyNPs.

### REFERENCE TO SEQUENCE LISTING SUBMITTED ELECTRONICALLY

The content of the electronically submitted sequence listing in ASCII text file (Name: Sequence_Listing.txt: Size: 21,959 bytes; and Date of Creation: Jul. 21, 2015) filed with the application is incorporated herein by reference in its entirety.

### FIELD OF THE INVENTION

The present invention relates to a novel enzymatic process for the production of cytosine Nucleoside Analogues (NAs), in particular for the industrial production of cytosinic NAs active as pharmaceutically relevant antiviral and anticancer drugs, intermediates or prodrugs thereof.

### BACKGROUND OF THE INVENTION

Nucleoside analogues (NAs) are synthetic compounds structurally related to natural nucleosides. In terms of their structure, nucleosides are constituted by three key elements: (i) the hydroxymethyl group, (ii) the heterocyclic nitrogenous base moiety, and (iii) the furanose ring, which in several instances seems to act as a spacer presenting the hydroxymethyl group and the base in the correct orientation.

NAs are characterized by great structural diversity, since they exhibit variously modified carbohydrate and/or aglycon fragments.

NAs are extensively used as antiviral and antitumor agents. These molecules have been traditionally synthesized by different chemical methods which often require time-consuming multistep processes including protection-deprotection reactions on the heterocycle base and/or the pentose moiety to allow the modification of naturally occurring nucleosides (Boryski J. 2008. Reactions of transglycosylation in the nucleoside chemistry. Curr Org Chem 12:309-325). These time consuming multistep processes often lead to low yields and increased costs. Indeed, chemical methods usually increase the difficulty of obtaining products with correct stereo- and regioselectivity, generating by-products as impurities (Condezo, L. A., et al. 2007. Enzymatic synthesis of modified nucleosides, p. 401-423. Biocatalysis in the pharmaceutical and biotechnology industries. CRC Press, Boca Raton, Fla., Mikhailopulo, I. A. 2007; Sinisterra, J. V. et al. 2010. Enzyme-catalyzed synthesis of nonnatural or modified nucleosides, p. 1-25. Encyclopedia of Industrial Biotechnology: Bioprocess, Bioseparation, and Cell Technology. John Wiley & sons, Ed. By M. C. Flickinger, 2010). Moreover, the chemical methods include the use of chemical reagents and organic solvents that are expensive and environmentally harmful.

Enzymatic synthesis of nucleosides involves the use of enzymes that catalyze the condensation of heterocyclic bases and sugars, thus forming glycoside bonds. In general terms, nucleoside analogs can be prepared by base interchange (transglycosilation reaction) using two different kind of enzymes: nucleoside phosphorylases (NPs or NP) and N-2′-deoxyribosyl transferases (NDTs or NDT). NPs include: Pyrimidine nucleoside phosphorylases (E.C. 2.4.2.1), Purine nucleoside phosphorylases (E.C. 2.4.2.2). Uridine nucleoside phosphorylases (E.C. 2.4.2.3) and Thymidine nucleoside phosphorylases (E.C. 2.4.2.4), according to their substrate specificity for pentofuranose donors and nucleobase acceptors. However, cytosine and its nucleosides (cytidine, 2′-deoxycytidine) or modified cytosinic analogues or derivatives and corresponding nucleosides thereof, are not substrates for these NPs enzymes (Mikhailopulo, I. A, et al. 2010. New Trends in Nucleoside Biotechnology. Acta Naturae, 2(5), 36-58).

Contrarily, N-2′-deoxyribosyl transferases (E.C. 2.4.2.6.) specifically catalyze the direct transfer of the deoxyribofuranosyl moiety between a nucleoside and an acceptor base without intermediary formation of 2-deoxyribofuranose phosphate. NDTs substrate specificity involves strict specificity for the 2-deoxyribofuranose moiety, rather than broad tolerance regarding modified purines and good substrate activity of cytosine as an acceptor of the 2-deoxy- and 2,3-dideoxyribofuranose residues (Mikhailopulo, I. A, et al. 2010. New Trends in Nucleoside Biotechnology, Acta Naturae, 2(5), 36-58). Fresco-Taboada et al (New insights on NDTs: a versatile Biocatalyst for one-pot one-step synthesis of nucleoside analogs, Appl. Microbiol. Biotechnol, 2013, 97, 3773-3785) disclose that cytosine is the best acceptor of 2′-deoxyribofuranose moiety in all cases.

Therefore, the choice of NPs or NDTs depends on the structure of the acceptor-base and the donor nucleoside. NPs and NDTs complement each other, allowing the biocatalytic production of natural nucleoside and their modified analogs, most of them useful for the production of many anticancer and antiviral drugs.

However, none of NPs or NDTs allow the production of cytosinic ribonucleosides, because cytosine is not an acceptor base for NPs while, at the same time, ribofuranose nucleoside donors are not substrates for NDTs. Therefore, the production of cytosinic nucleoside analogues remains a challenge, not only due to the above mentioned substrate specificity, but also because certain enzymes, such as cytosine and cytidine deaminases or cytosine and cytidine deacetylases that are usually present in the biocatalyst preparation, degrade the substrates or the final product, resulting in unproductive methodology for industrial purposes.

Araki et al (EP 1254959, U.S. Pat. No. 7,629,457) disclosed a method for producing cytosine nucleoside compounds from pentose-1-phosphate and cytosine or a derivative thereof using a nucleoside phosphorylase reactive to cytosine or a bacterium having said enzyme activity. Particularly, the inventors found that a purine nucleoside phosphorylase (PNP) from bacteria belonging to genus Escherichia, which itself should catalyze a reaction involving a purine base as substrate instead of a pyridine base, was able to catalyze the production of cytosine nucleoside from cytosine and pentose-1-phosphate. However, the reaction of cytosine or a derivative thereof with pentose-1-phosphate in the presence of the bacterium having the enzyme activity disclosed therein was found to produce almost exclusively the deaminated products of cytosine, or the derivative thereof, thus failing in efficient accumulation of the cytosine nucleoside compound of interest. In order to minimize the production of deaminated cytosine by-products, the referred patents additionally disclosed a method for specifically reducing the deaminase activity that degrade the substrates or the final product, by contacting the enzyme preparation with organic solvents and heating the enzyme preparation at a temperature from 60° C. to 90° C. for an effective period of time.

Araki et al (US 2005/0074857) disclosed a method for producing a pyrimidine nucleoside compound and a new pyrimidine nucleoside compound using a pyrimidine base derivative using an enzyme known as purine nucleoside phosphorylase (PNP) derived from a microorganism such as E. coli, the enzyme also having cytosine nucleoside phosphorylase activity. Authors overcome certain deacetylation deactivation by heating the bacterial cell of the microorganism or the processed enzyme or placing in contact with suitable organic solvents. The same authors disclosed several methods to produce the above mentioned compounds without applying a complicate deaminase-deactivating treatment, by producing a microorganism lacking both the cytosine deaminase gene and the cytidine deaminase gene (JP2004344029) or by means of a zinc salt (JP2004024086).

Ding Q. 2010. Enzymatic synthesis of nucleosides by mucleoside phosphorylase co-expressed in Escherichia coli. J. Zheijiang Univ-Sci B (Biomed & Biotechnol) 11 (11): 880-888, disclosed synthesis of many types of nucleosides. However, the disclosure failed in producing cytosine or arabinoside nucleosides.

Szeker, K. 2012. Nucleoside phosphorylases from thermophiles, Ph.D. Thesis, Berlin Institute of Biotechnology, Germany, disclose discloses that no phosphorolytic activity of GtPyNP (NP from Geobacillus thermoglucosidasius), only poor activity of TtPyNP (MP from Thermus thermophilus) were determined for cytidine as substrates (vide supra, p. 84) and cytidine was weakly recognized as substrate by PNPs and ApMTAP (5′-Methylthioadenosine phosphorylase from Aeropyrum pernix, vide supra, p. 116).

Thus, the biocatalytic synthesis of cytosinic nucleoside analogues remains a challenge and its application at industrial scale is limited by product degradation due to competitive enzymes that are present in the biocatalyst preparation, as explained above.

Since several non-natural nucleosides acting as antiviral or anticancer agents contain cytosine as nucleobase moiety, there is a need for the development of novel and effective industrial enzymatic processes that may overcome the before mentioned drawbacks and improve the synthesis of this type of cytosine nucleoside analogues.

Capecitabine, a well known anticancer agent having a cytosine NA chemical structure, it has been produced in the past, according to several processes described in the prior art for the multi-step chemical synthesis of this NA (the originators at F. Hoffmann-La Roche AG, Arasaki et al, EP0602454; Kamiya et al, EP0602478; see also U.S. Pat. No. 5,453,497).

The above processes suffer from certain drawbacks such as: the toxic solvents or reagents that are used, the need of a chromatographic purification step, which is unsuitable for large scale production, multi-step complex processes including protection and deprotection stages of the hydroxyl groups on the glycoside subunit, and low to moderate overall yields of capecitabine synthesis. The protection and deprotection sequence adds two extra steps in all these processes, then reducing the overall yield and increasing the time and cost of the process from a commercial production point of view.

In WO 2011/104540 a one step process for the preparation of Capecitabine is disclosed. The process is based on the reaction of 5-fluoro-5′-deoxycytidine with a pentyloxycarbonylation reagent that avoid the need for the conventional protection of the hydroxyl groups of the preformed nucleoside 5-fluoro-5′-deoxycytidine. The reaction takes place in organic solvents, uses chemical catalysts (such as bases or corrosive mineral acids as HCl gas), high temperatures (at solvent reflux, up to 110° C.) and long reaction times (usually 9 to 10 hours), rendering Capecitabine in moderate yields (50 to 67%).

Kanan et al (WO 2010/061402) disclose a process using (CALB) Novozyme 435 (a lipase acrylic resin from Candida antartica) as catalyst in the presence of organic solvent. Such process requires at least four steps to furnish capecitabine.

The same happens for Cytarabine synthesis. Cytarabine is another well known anti-cancer agent also sharing a cytosine NA chemical structure, as Capecitabine. There are early patents in the name of Merck (NL6511420 in 1964 or in the name of Salt lake Institute of Biological Studies (1969)), which already described chemical synthesis of said active compound, wherein the same drawbacks mentioned for Capecitabine might be equally mentioned.

Then, according to the above disadvantages associated with the prior art and the importance of capecitabine in the treatment of cancer, there is a great need to develop an improved process for the preparation of this Active Principle Ingredient (API) that does not involve, multiple steps, uses relatively inexpensive reagents and that it would render the nucleoside analogue product in high yield and purity.

### SUMMARY OF THE INVENTION

The presented disclosure provides methods for the enzymatic production of cytosinic nucleoside analogues, and methods for their use to treat diseases and conditions, for example, infections (e.g., viral infections) and cancer in a subject in need thereof. In some aspects, the subject is a human subject. In particular, the disclosure it relates to a new synthesis process of cytosine nucleoside analogues by using nucleoside phosphorylase enzymes, particularly Pyrimidin Nucleoside Phosphorylases (PyNPs) or mixtures of Purine Nucleoside Phosphorylases (PNPs) and PyNPs.

The disclosure provides a method for producing cytosine nucleoside analogues, intermediates or prodrugs thereof of formula I by chemo-enzymatic or enzymatic synthesis

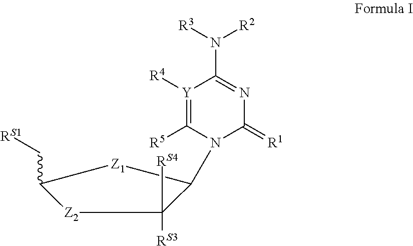

wherein,
Z₁ being O, CH₂, S, NH;
Z₂ being, independently of Z₁: O, C(R^{S2}R^{S5}), S(R^{S2}R^{S5}), S(R^{S2}), S(R^{S5}), preferably, a group SO or SO₂: N(R^{S2}R^{S5}), N(R^{S2}), N(R^{S5});
R^{S1} being hydrogen, OH, ether or ester thereof selected from:

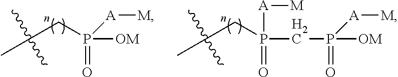

being n is 0 or 1, A is oxygen or nitrogen, and each M is independently hydrogen, an optionally substituted alkyl chain, an optionally substituted alkenyl chain, an optionally substituted alkynyl chain, an optionally substituted aryl optionally linked to P by an optionally substituted alkyl, alkenyl or alkynyl chain, an optionally substituted heterocycle optionally linked to P by an optionally substituted alkyl, alkenyl or alkynyl chain, or a pharmaceutically acceptable counter-ion such as, but not restricted to, sodium, potassium, ammonium or alkylammonium;
R^{S2} being hydrogen, OH or an ether or ester residue thereof, halogen, CN, NH₂, SH, C≡CH, N₃;
R^{S3} being hydrogen, in case of NA derived from 2′-deoxyribonucleosides or arabinonucleosides or being selected from: OH, NH₂, halogen, OCH₃, when the NA is derived from ribonucleosides;
R^{S3} being hydrogen, OH or an ether or ester residue thereof, NH₂, halogen, CN;
providing R^{S1} and R^{S4} are different when both were ethers or esters of OH residues;
R^{S5} being hydrogen, OH or an ether or ester residue thereof, NH₂ or halogen, in the case that Z₂ is different from oxygen;
R¹ being O, CH₂, S, NH;
R² being hydrogen, an optionally substituted C₄₋₄₀ alkyl chain, an optionally substituted alkenyl chain, an optionally substituted alkynyl chain, an optionally substituted aryl linked to N by an optionally substituted alkyl, alkenyl or alkynyl chain, an optionally substituted heterocycle linked to N by an optionally substituted alkyl, alkenyl or alkynyl chain, COR⁶, CONR⁶R⁷, CO₂R⁶, C(S)OR⁷, CN, SR⁶, SO₂R⁶, SO₂R⁶R⁷, CN, P(O)aryl, P(O)heterocycle, P(S)aryl, P(S)heterocycle, P(O)O₂R⁸;
R³ being hydrogen, an optionally substituted C₄₋₄₀ alkyl chain, an optionally substituted alkenyl chain, an optionally substituted alkynyl chain, an optionally substituted aryl linked to N by an optionally substituted alkyl, alkenyl or alkynyl chain, an optionally substituted heterocycle linked to N by an optionally substituted alkyl, alkenyl or alkynyl chain, COR⁶, CONR⁶R⁷, CO₂R⁶, C(S)OR⁷, CN, SR⁶, SO₂R⁶, SO₂R⁶R⁷, CN, P(O)aryl, P(O)heterocycle, P(S)aryl, P(S)heterocycle, P(O)O₂R⁸; being R¹ and R² independent one from each other; and providing that at least one R₂ or R₃ is different from hydrogen;
R₄ being hydrogen, OH, NH₂, SH, halogen; optionally substituted alkyl chain; optionally substituted alkenyl chain; optionally substituted alkynyl chain, trihaloalkyl, OR⁶, NR⁶R⁷, CN, COR⁶, CONR⁶R⁷, CO₂R⁶, C(S)OR⁶, OCONR⁶R⁷, OCO₂R⁶, OC(S)OR⁶, NHCONR⁶R⁷, NHCO₂R⁶, NHC(S)OR⁶, SO₂NR⁶R⁷, an optionally substituted aryl linked to Y by an optionally substituted alkyl, alkenyl or alkynyl chain, an optionally substituted heterocycle linked to Y by an optionally substituted alkyl, alkenyl or alkynyl chain, and any optionally substituted heterocycle or optionally substituted aryl of, independently, R², R³, R⁴ or R⁵, selected from:

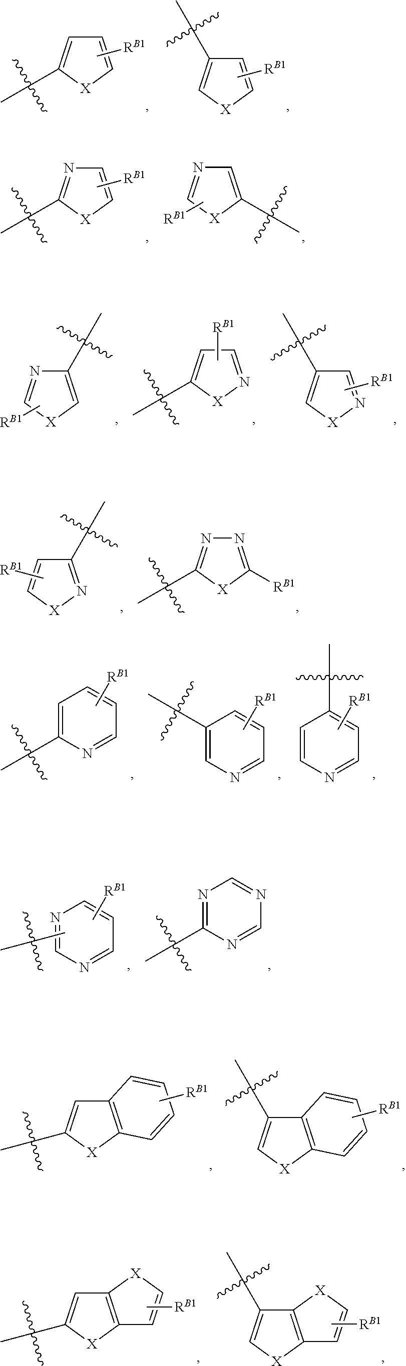

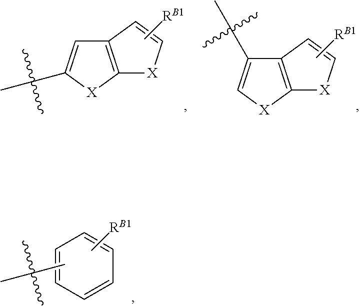

providing Y is a carbon or sulphur atom and, alternatively, R⁴ being absent, providing Y is a nitrogen atom;
wherein
X being O, S, N—R^{B2}, Se; R^{B1} being H, OH, NH₂, SH, straight or branched C₁₋₁₀ alkyl, F, Cl, Br, I, X—R^{B2}, —C≡C—R^{B2}, CO₂R^{B2}; R^{B2} being H, OH, NH₂, straight or branched C₁₋₅ alkyl, phenyl;
R⁵ being hydrogen, OH, NH₂, SH, halogen, an optionally substituted alkyl chain, an optionally substituted alkenyl chain, an optionally substituted alkynyl chain, trihaloalkyl, OR⁶, NR⁶R⁷, CN, COR⁶, CONR⁶R⁷, CO₂R⁶, C(S)OR⁶, OCONR⁶R⁷, OCO₂R⁶, OC(S)OR⁶, NHCONR⁶R⁷, NHCO₂R⁶, NHC(S)OR⁶, SO₂NR⁶R⁷; CH₂-heterocyclic ring, CN;
and any optionally substituted heterocycle or optionally substituted aryl of, independently, R², R³, R⁴ or R⁵, selected from:

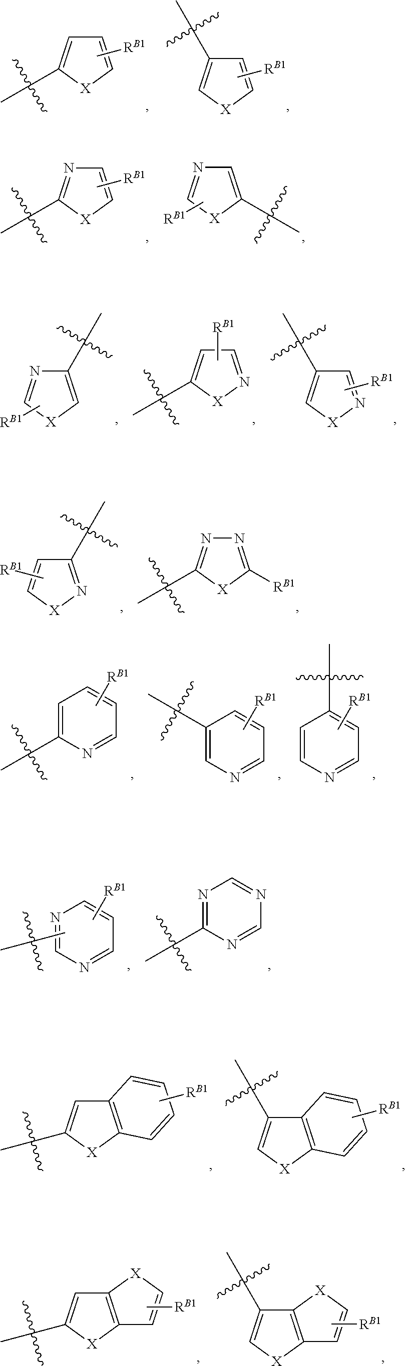

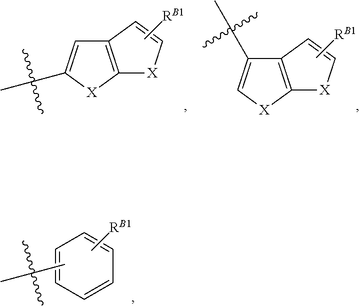

wherein
X being O, S, N—R^{B2}, Se; R^{B1} being H, OH, NH₂, SH, straight or branched C₁₋₁₀ alkyl, F, Cl, Br, I, X—R^{B2}, —C≡C—R^{B2}, CO₂R^{B2}; R^{B2} being H, OH, NH₂, straight or branched C₁₋₅ alkyl, phenyl;
R⁶ and R⁷ are independently of each other hydrogen, optionally substituted alkyl chain, optionally substituted alkenyl chain, optionally substituted alkynyl chain, heterocyclic or optionally substituted aryl;
R⁸ being hydrogen, an optionally substituted alkyl chain, an optionally substituted alkenyl chain, an optionally substituted alkynyl chain, an optionally substituted aryl an optionally substituted heterocycle;
Y being C, N, S;
wherein the method comprises:
(i) chemically reacting a precursor of the cytosinic nucleobase of formula II, wherein Y, R¹, R⁴, R⁵, R⁶ and R⁷, are defined as above, with suitable reagents for modifying its amino group at N⁴ position in order to incorporate the proper substitution described as substituent R² and R³, said modified cytosinic nucleobase of formula II formed thereof, being optionally purified by conventional purification methods; or alternatively, the process departs directly from the starting products represented by a cytosinic nucleobase of formula II as such.

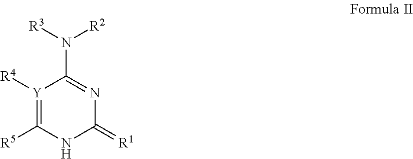

(ii) biocatalytically reacting the above mentioned modified cytosinic nucleobase of formula H, with a suitable nucleoside analog substrate of formula III,

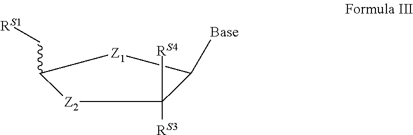

wherein Z₁, Z₂, R^{S1}, R^{S2}, R^{S3}, R^{S4}, R^{S5} are defined as above and the Base is selected from: uracil, adenine, cytosine, guanine, thymine, hypoxanthine, xanthine, thiouracil, thioguanine, 9-H-purine-2-amine, 7-methylguanine, 5-fluorouracil, 5-bromouracil, 5-chlorouracil, 5,6-dihydrouracil, 5-methylcytosine and 5-hydroxymethylcytosine, pteridone, and any substituted derivative thereof;
wherein the aforesaid reaction carried out in step ii), comprises the addition, in a suitable reaction aqueous medium and under suitable reaction conditions, of a nucleoside phosphorylase enzyme, either a pyrimidine nucleoside phosphorylase enzyme, a purine nucleoside phosphorylase enzyme or combinations thereof, to a mixture of starting materials comprising a cytosine nucleobase of formula II and a nucleoside analogue of formula III,
(iii) optionally, deprotecting the amino group at N⁴ position in cytosinic nucleoside analogue in order to recover the free primary amino N⁴ at the cytosinic nucleoside analogue of formula I which was further purified by conventional purification methods.

In some aspects, the cytosinic nucleobase of formula II to be transferred by the pyrimidine nucleoside phosphorylase enzyme is selected from:

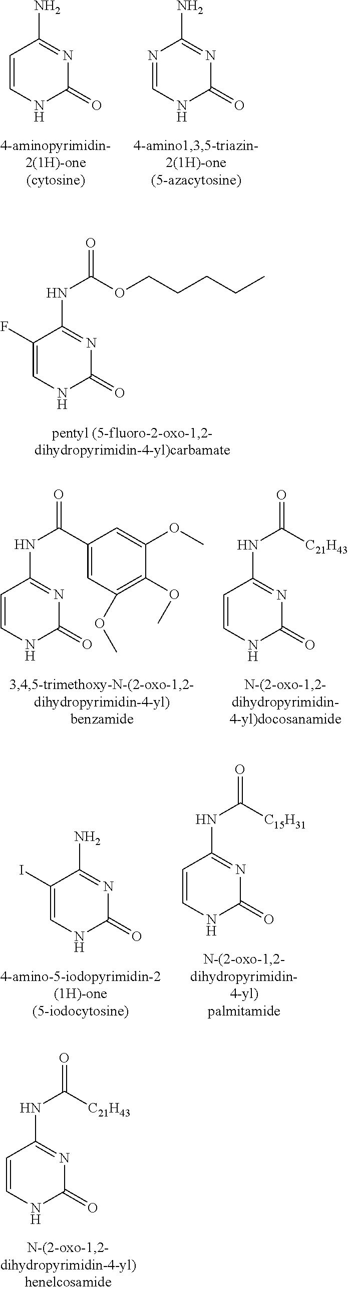

In some aspects, the nucleoside analogs, intermediates or prodrugs thereof produced are selected from: Capecitabine, Decitabine, 5-Azacytidine, Cytarabine, Enocitabine, Gemcitabine, Zalcitabine, Ibacitabine, Sapacitabine, 2′-C-cyano-2′-deoxy-1-β-D-arabino-pentofuranosylcytosine, Galocitabine, Valopricitabine, 2′-Deoxy-4′-thiocytidine, Thiarabine, 2′-Deoxy-4′-thio-5-azacytidine and Apricitarabine.

In some aspects, the source of native, mutants or variants thereof, or of recombinant nucleoside phosphorylase enzyme, are mesophilic, thermophilic or hyperthermophilic organisms.

In some aspects, the source of native and mutants or variants thereof, or of recombinant nucleoside phosphorylase enzyme is selected from Archaea or bacteria.

In some aspects, the nucleoside phosphorylase enzyme is isolated from an Archaea selected from: Sulfolobus solfataricus or Aeropyrum pernix.

In some aspects, the nucleoside phosphorylase enzyme, or a functional part thereof, is encoded by a nucleotide sequence selected from: SEQ ID NO. 1, 2, 5, 7, 9 or 11; or
a) a nucleotide sequence which is the complement of SEQ ID. NO: 1, 2, 5, 7, 9 or 11; or
b) a nucleotide sequence which is degenerate with SEQ ID. NO: 1, 2, 5, 7, 9 or 11; or
c) a nucleotide sequence hybridizing under conditions of high stringency to SEQ ID. NO:1, 2, 5, 7, 9 or 11; to the complement of SEQ ID. NO:1, 2, 5, 7, 9 or 11; or to a hybridization probe derived from SEQ ID. NO: 1, 2, 5, 7, 9 or 11; or their complement thereof; or
d) a nucleotide sequence having at least 80% sequence identity with SEQ ID. NO:1, 2, 5, 7, 9 or 11; or
e) a nucleotide sequence having at least 59% sequence identity with SEQ ID. NO: 1, 2.5, 7, 9 or 11; or
f) a nucleotide sequence encoding for an amino acid sequence selected from: SEQ ID. NO: 3, 4, 6, 8, 10 or 12.

In some aspects, the nucleoside analogue produced is, Capecitabine, the ribonucleoside used as starting material is selected from: 5′-deoxyuridine, 5′-deoxy-5-methyluridine or 5′-deoxy-5-chlorouridine; and the nucleobase used as starting material to be transferred by the Pyrimidine Nucleoside Phosphorilase enzyme, is pentyl (5-fluoro-2-oxo-1,2-dihydropyrimidin-4-yl)carbamate.

In some aspects, the nucleoside analogue produced is, Cytarabine, the ribonucleoside used as starting material is 9-(b-D-arabinofuranosyl)uracil and the nucleobase used as starting material to be transferred by the Pyrimidine Nucleoside Phosphorilase enzyme, is N-(2-oxo-1,2-dihydropyrimidin-4-yl) pentanamide.

Also provided is a method to produce a cytosinic nucleoside analogue, intermediate or prodrug thereof for the treatment of cancer or viral infection in a subject in need thereof comprising the use of
(a) a native mesophilic, thermophilic or hyperthermophilic nucleoside phosphorylase, or a combination thereof;
(b) a mutant mesophilic, thermophilic or hyperthermophilic nucleoside phosphorylase, or a combination thereof;
(c) a variant mesophilic, thermophilic or hyperthermophilic nucleoside phosphorylase, or a combination thereof;
(d) a recombinant mesophilic, thermophilic or hyperthermophilic nucleoside phosphorylase, or a combination thereof;
(e) a functional fragment of a mesophilic, thermophilic or hyperthermophilic nucleoside phosphorylase, or a combination thereof;
(f) a recombinant expression vector comprising a sequence encoding a nucleoside phosphorylase according to (a), (b), (c), (d) or (e), operably linked to one or more control sequences that direct the expression or overexpression of said nucleoside phosphorylase in a suitable host; or
(g) a microorganism or a host cell containing (a), (b), (c), (d), (e) or (f) or a combination thereof.

Also provided are methods to treat an infection (e.g., a viral infection) or cancer in subject in need thereof comprising the administration of a compound or formulation comprising a cytosinic nucleoside analogue, intermediate or prodrug obtained using a manufacturing process which comprises the use of
(a) a native mesophilic, thermophilic or hyperthermophilic nucleoside phosphorylase, or a combination thereof;
(b) a mutant mesophilic, thermophilic or hyperthermophilic nucleoside phosphorylase, or a combination thereof;
(c) a variant mesophilic, thermophilic or hyperthermophilic nucleoside phosphorylase, or a combination thereof;
(d) a recombinant mesophilic, thermophilic or hyperthermophilic nucleoside phosphorylase, or a combination thereof;
(e) a functional fragment of a mesophilic, thermophilic or hyperthermophilic nucleoside phosphorylase, or a combination thereof;
(f) a recombinant expression vector comprising a sequence encoding a nucleoside phosphorylase according to (a), (b), (c), (d) or (e), operably linked to one or more control sequences that direct the expression or overexpression of said nucleoside phosphorylase in a suitable host; or
(g) a microorganism or a host cell containing (a), (b), (c), (d), (e) or (f) or a combination thereof.

In some aspects, the mesophilic, thermophilic or hyperthermophilic nucleoside phosphorylase enzyme, or a functional part thereof in the method to produce or the method of treatment disclosed above, is encoded by a nucleotide sequence selected from: SEQ ID NO. 1, 2, 5, 7, 9 or 11; or
a) a nucleotide sequence which is the complement of SEQ ID. NO:1, 2, 5, 7, 9 or 11; or
b) a nucleotide sequence which is degenerate with SEQ ID. NO:1, 2, 5, 7, 9 or 11; or
c) a nucleotide sequence hybridizing under conditions of high stringency to SEQ ID. NO:1, 2, 5, 7, 9 or 11; to the complement of SEQ ID. NO:1, 2, 5, 7, 9 or 11; or to a hybridization probe derived from SEQ ID. NO: 1, 2, 5, 7, 9 or 11; or their complement thereof; or
d) a nucleotide sequence having at least 80% sequence identity with SEQ ID. NO: 1, 2.5, 7, 9 or 11; or
e) a nucleotide sequence having at least 59% sequence identity with SEQ ID. NO: 1, 2, 5, 7, 9 or 11; or
f) a nucleotide sequence encoding for an amino acid sequence selected from: SEQ ID. NO: 3, 4, 6, 8, 10 or 12.

In some aspects, the cytosinic nucleoside analogues, intermediates or prodrugs thereof in the method to produce or the method of treatment disclosed above are selected from: Capecitabine, Decitabine, 5-Azacytidine, Cytarabine, Enocitabine, Gemcitabine, Zalcitabine, Ibacitabine, Sapacitabine, 2′-C-cyano-2′-deoxy-1-β-D-arabino-pentofuranosylcytosine, Galocitabine, Valopricitabine, 2′-Deoxy-4′-thiocytidine, Thiarabine, 2′-Deoxy-4′-thio-5-azacytidine and Apricitarabine. In some specific aspects, the cytosinic nucleoside analogue, intermediate or prodrug thereof produced is Capecitabine. In some aspects, the cytosinic nucleoside analogue, intermediate or prodrug thereof produced is Cytarabine.

### DETAILED DESCRIPTION OF THE INVENTION

The drawbacks of biocatalytic synthesis methods known in the art can be avoided by applying a suitable enzymatic method based on the use of Nucleoside Phosphorylases (NPs), either Purine Nucleoside Phosphorylases (PNPs), or Pyrimidine Nucleoside Phosphorylases (PyNP), or a mixture of Pyrimidine Nucleoside Phosphorylases (PyNP) and Purine Nucleoside Phosphorylases (PNP) enzymes. As a result cytosinic NAs can be obtained with conversion higher than 70% and anomeric purity higher than 95%. The referred enzymes can recognize proper chemically modified cytosines and are able to perform the transglycosilation reaction on donor nucleoside analogs without any evidence of deamination or deacetylation impurities.

For the purposes of present specification, enzymes to be used in the process of invention are, for example, Pyrimidine Nucleoside Phosphorylases. Another embodiment of present invention is the use of a mixture of Pyrimidine Nucleoside Phosphorylases (PyNP) and Purine Nucleoside Phosphorylases (PNP) enzymes, particularly when, as starting compound, a purine nucleoside or derivatives or analogs thereof, are used.

The process of invention applied, as a way of example, to the production of Capecitabine or Cytarabine, as cytosine NAs, has several advantages over the processes used in the prior art for the chemical synthesis of these NAs, and also with regard to the enzymatic synthesis using CALB Novozyme 435, such as:
(i) Reduced number of steps,
(ii) Higher conversions and yields,
(iii) No protection/deprotection strategies for the hydroxyl groups in the sugar are needed,
(iv) Mild reaction conditions: environmentally-friendly technology (water or aqueous medium, neutral pH),
(v) Avoidance of organic solvents in the enzymatic step.
(vi) Extremely good selectivity: stereoselectivity-enantioselectivity, chemo-regioselectivity,
(vii) No side-reactions: impurity profile (reduced by-products content),
(viii) Reduction in overall waste generation,
(ix) Process productivity
(x) Overall lower cost of production

Moreover, there are other additional advantages of the biocatalytic process of invention over the chemical process used in the prior art. Particularly relevant is the absence in the process of the invention of the organic solvents used in the chemical process of Arasaki et al. and Kamiya et al., as pyridine, dichloromethane (DCM), acetonitrile (ACN), methanol, tetrahydrofuran (THF), or in the enzymatic process of Kannan et al (pyridine, dichloromethane (DCM), etc).

Particularly relevant it is also the absence in the process of the invention of metal catalyst such as stannic chloride, toxic reagents such as sylilating agents, etc. All those process organic solvents and reagents must be removed or destroyed prior to any waste discharge to the environment. A supplementary inconvenient of processes described in the prior art with regard to the procedure of invention is its complexity, as far as processes described in the prior art comprise multi-step procedures including protection and deprotection steps, against the simpler enzymatic process of invention.

The one step process for the preparation of Capecitabine disclosed in WO 2011/104540 suffers from important loses of the final product. Capecitabine suffers from thermal instability at high temperatures, and after heating the product at 90° C. during 10 hours at neutral pH, up to 40% of the product is lost. On the contrary, the enzymatic process of the invention runs at milder conditions, using water as solvent and furnishing yields higher than 70%.

The process of invention as described herein using, for example, PyNP or PyNP/PNP enzymes, is outlined as follows:

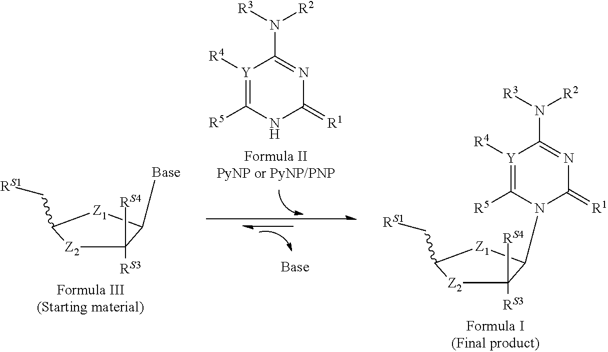

### Scheme 1

### Enzymatic Synthesis of Cytosinic Nucleoside Analogs

Applicants have found that suitable N⁴-modified cytosine or N⁴-modified cytosine derivatives allow the preparation/production of cytosinic nucleoside analogues at high conversions (more than 70%), completely avoiding side reactions, such as the deamination or deacetylation that are reported in the prior art cited previously, by using nucleoside phosphorylases (either native, recombinant or mutant proteins from bacteria or archaea), and particularly using pyrimidine nucleoside phosphorylases (either native, recombinant or mutant proteins from bacteria or archaea).

No evidences have been found in the prior art pointing at the fact that a chemical modification/substitution/protection at this position or in any other position in cytosine chemical backbone, would have been able to modify the substrate specificity for nucleoside phosphorylases. For the purposes of present specification the term N⁴-modified cytosine/cytosine derivatives, are synonyms, respectively, of either N⁴-substituted cytosine/cytosine derivatives or N⁴-protected cytosine/cytosine derivatives.

For the purposes of present patent specification the term Cytosine or cytosinic derivatives, nucleosides, intermediates, bases or nucleobases, they all should be understood as chemical compounds derived from cytosine backbone. Particularly as the cytosine or cytosinic derivatives for the purposes of present invention are represented by formula II:

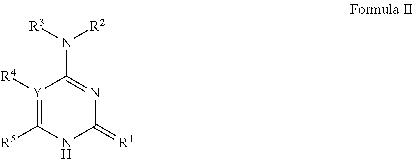

The suitable chemical modifications that are the object of the present invention provide a more convenient, efficient and easier process for nucleoside analogues synthesis that fully avoids the instability problem related to side reactions. Particular N⁴-modifications include, for example, the modification of the amino group in the form of carbamate (—NHCOO—), more particularly the carbamate being linked to an alkyl chain of 1 to 40 carbon atoms, an alkenyl chain of 1 to 40 carbon atoms or an alkynyl chain of 1 to 40 carbon atoms (the chains being in each case lineal, branched, or substituted by any other functional group), an aryl or and heterocycle. N⁴-modifications of the amino group in the form of amide or related acyl derivatives (—NHCO—) allow the transglycosilation reaction as well, specially when the amido group is being linked to an alkyl chain of 4 to 40 carbon atoms, an alkenyl chain of 1 to 40 carbon atoms or an alkynyl chain of 1 to 40 carbon atoms (the chains being in each case lineal, branched, or substituted by any other functional group), an aryl or and heterocycle. Longer alkyl chains are preferred over short alkyl chains (from 1 o 3 carbon atoms), since according to the experiments carried out, deacetylation side reactions are also observed in short alkyl chains, differing from what US 2005/0074857 teaches away. According to present invention, acyl groups having an alkyl group of 1 carbon atom do not prevent deacetylation.

More precisely chemical modifications are introduced, according to present description, into cytosine backbone at N⁴ (Nitrogen atom at position 4 in the cytosine heterocycle). More particularly those chemical modifications operated in cytosine skeleton are the ones represented by R₂ and/or R₃ moieties in formula II:

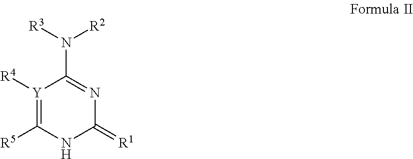

wherein
R¹ being O, CH₂, S, NH;
R² being hydrogen, an optionally substituted C₄₋₄₀ alkyl chain, an optionally substituted alkenyl chain, an optionally substituted alkynyl chain, an optionally substituted aryl linked to N by an optionally substituted alkyl, alkenyl or alkynyl chain, an optionally substituted heterocycle linked to N by an optionally substituted alkyl, alkenyl or alkynyl chain, COR⁶, CONR⁶R⁷, CO₂R⁶, C(S)OR⁷, CN, SR⁶, SO₂R⁶, SO₂R⁶R⁷, CN, P(O)aryl, P(O)heterocycle, P(S)aryl, P(S)heterocycle, P(O)O₂R⁸;
R³ being hydrogen, an optionally substituted C₄₋₄₀ alkyl chain, an optionally substituted alkenyl chain, an optionally substituted alkynyl chain, an optionally substituted aryl linked to N by an optionally substituted alkyl, alkenyl or alkynyl chain, an optionally substituted heterocycle linked to N by an optionally substituted alkyl, alkenyl or alkynyl chain, COR⁶, CONR⁶R⁷, CO₂R⁶, C(S)OR⁷, CN, SR⁶, SO₂R⁶; SO₂R⁶R⁷, CN, P(O)aryl, P(O)heterocycle, P(S)aryl, P(S)heterocycle, P(O)O₂R⁸; being R³ and R² independent one from each other, and providing that at least one R₂ or R₃ is different from hydrogen.
R⁴ being hydrogen, OH, NH₂, SH, halogen (preferably F or I); optionally substituted alkyl chain; optionally substituted alkenyl chain; optionally substituted alkynyl chain, trihaloalkyl, OR⁶, NR⁶R⁷, CN, COR⁶, CONR⁶R⁷, CO_{2R}⁶, C(S)OR⁶, OCONR⁶R⁷, OCO₂R⁶, OC(S)OR⁶, NHCONR⁶R⁷, NHCO₂R⁶, NHC(S)OR⁶, SO₂NR⁶R⁷, an optionally substituted aryl linked to Y by an optionally substituted alkyl, alkenyl or alkynyl chain, an optionally substituted heterocycle linked to Y by an optionally substituted alkyl, alkenyl or alkynyl chain, and any optionally substituted heterocycle or optionally substituted aryl of, independently, R¹, R², R³, R⁴ or R⁵, selected from:

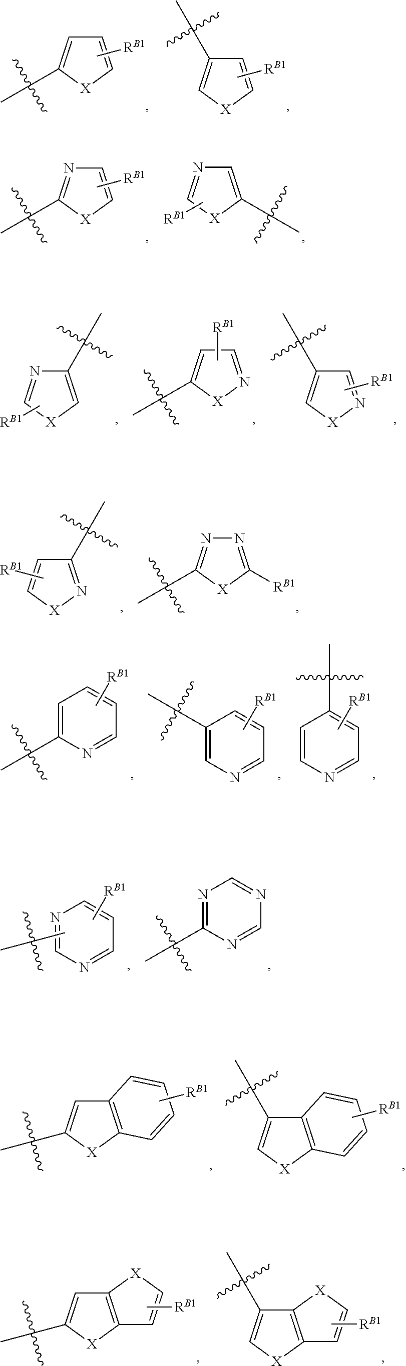

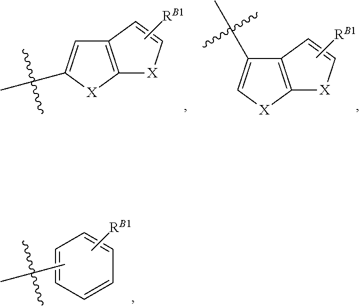

providing Y is a carbon or sulphur atom and, alternatively, R⁴ being absent, providing Y is a nitrogen atom;
wherein
X being O, S, N—R^{B2}, Se; R^{B1} being H, OH, NH₂, straight or branched C₁₋₁₀ alkyl, F, Cl, Br, I, X—R^{B2}, —C≡C—R^{B2}, CO₂R^{B2}; R^{B2} being H, OH, NH₂, straight or branched C₁₋₅ alkyl, phenyl;
R⁵ being hydrogen, OH, NH₂, SH, halogen (preferably F or I), an optionally substituted alkyl chain, an optionally substituted alkenyl chain, an optionally substituted alkynyl chain, trihaloalkyl, OR⁶, NR⁶R⁷, CN, COR⁶, CONR⁶R⁷, CO₂R⁶, C(S)OR⁶, OCONR⁶R⁷, OCO₂R⁶, OC(S)OR⁶, NHCONR⁶R⁷, NHCO₂R⁶, NHC(S)OR⁶, SO₂NR⁶R⁷; CH₂-heterocyclic ring, CN;
and any optionally substituted heterocycle or optionally substituted aryl of, independently, R², R³, R⁴ or R⁵, selected from:

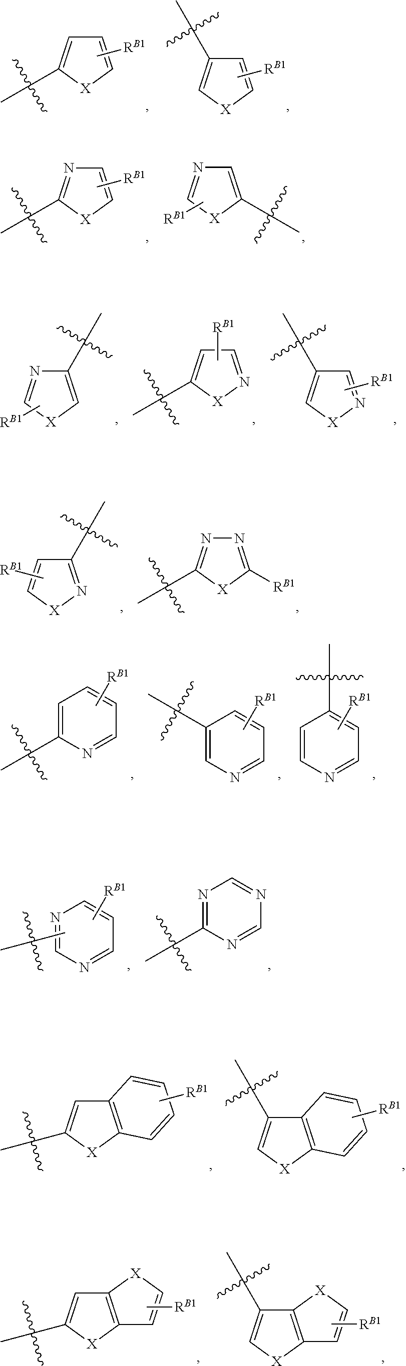

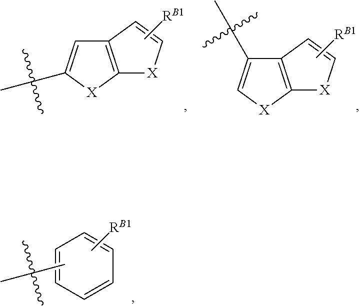

wherein
X being O, S, N—R^{B2}, Se; R^{B1} being H, OH, NH₂, SH, straight or branched C₁₋₁₀ alkyl, F, Cl, Br, I, X—R^{B2}, —C≡C—R^{B2}, CO₂R^{B2}; R^{B2} being H, OH, NH₂, straight or branched C₁₋₅ alkyl, phenyl;
R⁶ and R⁷ are independently of each other hydrogen, optionally substituted alkyl chain, optionally substituted alkenyl chain, optionally substituted alkynyl chain, heterocyclic or optionally substituted aryl;
R⁸ being hydrogen, an optionally substituted alkyl chain, an optionally substituted alkenyl chain, an optionally substituted alkynyl chain, an optionally substituted aryl an optionally substituted heterocycle;
Y being C, N, S;

According to the comparative examples carried out by the applicants and shown in present description, cytosine derivatives that do not contain suitable modifications at N⁴ do not undergo the transglycosilation reaction using nucleoside phosphorylases (native, recombinant or mutant enzymes), particularly using pyrimidine nucleoside phosphorylases, and therefore, do not render the desired nucleoside final product.

Moreover, non-protected cytosines (either bases or the corresponding nucleosides) experienced side reactions (such as deamination or deacetylation), that some authors try to deactivate by conventional methods such as heating or using organic solvents (EP1254959 and US2005/0074857), adding zinc salts (JP2004024086) or by using more sophisticated methods, such as using a microorganism simultaneously lacking both the cytosine deaminase gene and the cytidine deaminase gene (JP2004344029). All the above mentioned technical solutions disclosed in the prior art, lowered drastically the overall yield of the corresponding synthesis process. Present invention provides a synthesis method of nucleoside analogues to completely and definitively resolve the above referred technical problems concerning side-reactions that diminished the overall yield of the synthesis process described herein.

The functional group used for modifying/substituting/protecting N⁴ position in the final product obtained, can be optionally removed (deprotection of final products), according to the chemical structure of, specifically, each final product to be produced. However, in certain cases, such as for Capecitabine molecule, there is no need to deprotect the modified/substituted/protected cytosinic N⁴ group, since the modification remains attached to the amino group in the final product (or API). For Capecitabine itself, the enzymatic method of invention shortens, to a large extend, the time needed when the chemical conventional production processes are used.

Hence, the invention provides improved alternative synthesis methods of nucleoside analogues, useful as anticancer and/or antiviral products, by shortening conventional multi-step synthesis, increasing overall yield, reducing side reactions and by-product content and, therefore, improving product purity and quality.

For the purposes of present description, the following terms are further defined as follows.

The term “nucleoside” refers to all compounds in which a heterocyclic base is covalently coupled to a sugar. In some aspects, coupling of the nucleoside to the sugar includes a C1′-(glycosidic) bond of a carbon atom in a sugar to a carbon- or heteroatom (typically nitrogen) in the heterocyclic base. Therefore, in the present context the term “nucleoside” means the glycoside of a heterocyclic base. Similarly, the term “nucleotide” refers to a nucleoside wherein a phosphate group is coupled to the sugar.

The term “nucleoside” can be used broadly as to include non-naturally occurring nucleosides, naturally occurring nucleosides as well as other nucleoside analogues. Illustrative examples of nucleosides are ribonucleosides comprising a ribose moiety as well as deoxyribonucleosides comprising a deoxyribose moiety. With respect to the bases of such nucleosides, it should be understood that this can be any of the naturally occurring bases, e.g. adenine, guanine, cytosine, thymine, and uracil, as well as any modified variants thereof or any possible unnatural bases.

The term “nucleoside analogue”, “nucleoside analog”, “NA” or “NAs” as used herein refers to all nucleosides in which the sugar is, preferably, not ribofuranose or arabinofuranose and/or in which the heterocyclic base is not a naturally occurring base (e.g., A, G, C, T, I, etc.).

As further used herein, the term “sugar” refers to all carbohydrates and derivatives thereof, wherein particularly contemplated derivatives include deletion, substitution or addition or a chemical group or atom in the sugar. For example, especially contemplated deletions include 2′-deoxy, 3′-deoxy, 5′-deoxy and/or 2′,3′-dideoxy-sugars. Especially contemplated substitutions include replacement of the ring-oxygen with sulphur or methylene, or replacement of a hydroxyl group with a halogen, azido, amino-, cyano, sulfhydryl-, or methyl group, and especially contemplated additions include methylene phosphonate groups. Further contemplated sugars also include sugar analogues (i.e., not naturally occurring sugars), and particularly carbocyclic ring systems. The term “carbocyclic ring system” as used herein refers to any molecule in which a plurality or carbon atoms form a ring, and in especially contemplated carbocyclic ring systems the ring is formed from 3, 4, 5, or 6 carbon atoms.

The term “chemo-enzymatic synthesis” refers to a method of synthesis of chemical compounds through a combination of chemical and biocatalytic steps. For the particular purposes of the present invention, the order of the reaction stages, in one of the embodiments of the invention must be, for example, 1) chemical modification of the cytosinic base: 2) biocatalytic transglycosilation, using a NP enzyme; 3) optionally further deprotection of the cytosine-N⁴ in the final product.

The term “enzymatic synthesis” refers to a method of synthesis of chemical compounds by means of a process which only comprises biocatalytic steps, carried out by the appropriate enzymes (NPs). Accordingly, other embodiment of the synthesis process described herein is a full biocatalytic process which departs from cytosine derivatives, as the ones previously mentioned as represented by general formula II, already prepared or available in the market as cytosine derivatives as such, particularly those showing R₂ and/or R₃ at N⁴ position of cytosine heterocyclic ring, according to present invention, and, therefore, omitting step 1 of chemical modification of the aforesaid cytosine backbone.

The terms “heterocyclic ring” or “heterocyclic base” or “base” or “nucleobase” are used interchangeably herein and refer to any compound in which plurality of atoms form a ring via a plurality of covalent bonds, wherein the ring includes at least one atom other than a carbon atom. Particularly contemplated heterocyclic bases include 5- and 6-membered rings containing at least 1 to 4 heteroatoms each independently selected from nitrogen, oxygen and sulphur as the non-carbon atom (e.g., imidazole, pyrrole, triazole, dihydropyrimidine). Further contemplated heterocycles can be fused (i.e., covalently bound) to another ring or heterocycle, and are thus termed “fused heterocycle” or “fused heterocyclic base” as used herein. Especially contemplated fused heterocycles include a 5-membered ring fused to a 6-membered ring (e.g., purine, pyrrolo[2,3-d]pyrimidine), and a 6-membered ring fused to another 6-membered or higher ring (e.g., pyrido[4,5-d]pyrimidine, benzodiazepine). Examples of these and further heterocyclic bases are given below. Still further contemplated heterocyclic bases can be aromatic, or can include one or more double or triple bonds. Moreover, contemplated heterocyclic bases and fused heterocycles can further be substituted in one or more positions. And any one of the rings being optionally substituted with one, two or three substituents each independently selected from the group consisting of halogen, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, polyhaloC₁₋₆alkoxy, and C₃₋₇cycloalkyl.

The terms “nucleobase” covers naturally occurring nucleobases as well as non-naturally occurring nucleobases. It should be clear to the person skilled in the art that various nucleobases which previously have been considered “non-naturally occurring” have subsequently found in nature. Thus, “nucleobase” includes not only the known purine and pyrimidine heterocycles, but also heterocyclic analogues (such as N-substituted heterocycles) and tautomers thereof. Illustrative examples of nucleobases are adenine, guanine, thymine, cytosine, uracil, purine, xanthine, 2-chloroadenine, 2-fluoroadenine, pentyl (5-fluoro-2-oxo-1,2, dihydropyrimidin-4-yl)carbamate, cytosine N-alkyl carbamates, cytosine N-alkylesters, 5-azacytosine, 5-bromovinyluracil, 5-fluorouracil, 5-trifluoromethyluracil, 6-methoxy-9H-purin-2-amine and (R)-3,6,7,8-tetrahydroimidazo[4,5-d][1,3]diazepin-8-ol.

The term “nucleobase” is intended to cover every and all of these examples as well as analogues and tautomers, and regioisomers thereof. In order to differentiate these “nucleobases” from other heterocyclic bases also present in this specification, for the purposes of present specification, the term “nucleobase” mainly refers to cytosinic bases represented by formula II. However, the term “bases”, for the purposes also of present specification, mainly relates to the bases present in the nucleosides represented by formula III.

The term “tautomer” or “tautomeric form” refers to structural isomer of different energies which are interconvertible via a low energy barrier. For example, proton tautomers (also known as prototropic tautomers) include interconversion via migration of a proton, such as keto-enol and imine-enamine isomerizations. Valence tautomers include interconversions by reorganization of some of the bonding electrons.

The term “regioisomer” refers to structural isomer, or constitutional isomer in the sense that refers to molecules with the same molecular formula that whose atoms are bonded in different order of connectivity.

The term “conversion” refers to is the percentage of starting material that is transformed into products, either the expected final product, byproducts, or even into products of degradation.

The term “yield” is the number of synthesized molecules of product per number of starting molecules. In a multistep synthesis, the yield can be calculated by multiplication of the yields of all the single steps.

The term “anomeric purity” refers to the amount of a particular anomer of a compound divided by the total amount of all anomers of that compound present in the mixture multiplied by 100%.

The term “cytosine modification/substitution/protection” refers to any nucleoside analogue in which at least one position on the original backbone of cytosine (except nitrogen at position 1) is substituted by a functional group such as those described as radicals R¹, R², R¹, R⁴, R⁵, X and/or Y, each one of these groups being independent from the others.

The term “cytosine modified/substituted/protected at position N⁴” refers to bases with cytosine backbone in which at least one proton of the amino group at position 4 is substituted by a functional group, such as those described as radicals R₂ and/or R₃, each one of these substituents being independent from the others, providing that at least one of the aforesaid substituents is different from hydrogen.

The term “intermediate” or “intermediates” refer to any nucleoside analogue type compounds which can be transformed into an active pharmaceutical ingredient (API) of nucleosidic structure by means of suitable additional chemical reactions. Therefore, intermediates are molecules that can be considered as API precursors. For the purposes of present specification the term “precursor” when applied to the N⁴-modified cytosine or cytosine derivatives or intermediates, is meant by compounds of formula II wherein moieties bound to N⁴ are chemical groups other than those represented by R² or R³.

The term “prodrug” as used throughout this text means the pharmacologically acceptable derivatives such as esters, amides, carbamates and phosphates, such that the resulting in vivo biotransformation product of the derivative is the active drug as defined in the compounds of formula (I). The reference by Goodman and Gilman (The Pharmacological Basis of Therapeutics, 8th ed, McGraw-Hill, Int. Ed. 1992, “Biotransformation of Drugs”, p 13-15) describing prodrugs generally is hereby incorporated. Prodrugs preferably have excellent aqueous solubility, increased bioavailability and are readily metabolized into the active inhibitors in vivo. Prodrugs of a compound of the present invention can be prepared by modifying functional groups present in the compound in such a way that the modifications are cleaved, either by routine manipulation or in vivo, to the parent compound.

In some aspects of the present invention, prodrugs are pharmaceutically acceptable ester, amide and carbamate that are hydrolysable in vivo and are derived from those compounds of formula I having a hydroxy or a amino group. An in vivo hydrolysable ester, amide and carbamate is an ester, amide or carbamate group which is hydrolysed in the human or animal body to produce the parent acid or alcohol. Suitable pharmaceutically acceptable esters, amide and carbamates for amino group include C₁₋₆alkoxymethyl esters for example methoxymethyl, C₁₋₆ alkanoyloxymethyl esters for example pivaloyloxymethyl, phthalidyl esters, C₃₋₈ cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyl-oxyethyl which can be formed at any carboxy group in the compounds of this invention.

An in vivo hydrolysable ester of a compound of the formula (I) containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

For therapeutic use, salts of the compounds of formula (I) are those wherein the counter-ion is pharmaceutically acceptable. However, salts of acids and bases which are non-pharmaceutically acceptable can also find use, for example, in the preparation or purification of a pharmaceutically acceptable compound. All salts, whether pharmaceutically acceptable or not are included within the scope of the present invention.

The pharmaceutically acceptable acid and base addition salts as mentioned above are meant to comprise the therapeutically active non-toxic acid and base addition salt forms which the compounds of formula (I) are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids.

Conversely said salt forms can be converted by treatment with an appropriate base into the free base form.

The compounds of formula (I) containing an acidic proton can also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term addition salt as used hereinabove also comprises the solvates which the compounds of formula (I) as well as the salts thereof, are able to form. Such solvates are for example hydrates, alcoholates and the like.

The term “quaternary amine” as used hereinbefore defines the quaternary ammonium salts which the compounds of formula (I) are able to form by reaction between a basic nitrogen of a compound of formula (I) and an appropriate quaternizing agent, such as, for example, an optionally substituted alkylhalide, arylhalide or arylalkylhalide, e.g. methyliodide or benzyliodide. Other reactants with good leaving groups can also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

The N-oxide forms of the present compounds are meant to comprise the compounds of formula (I) wherein one or several nitrogen atoms are oxidized to the so-called N-oxide.

It will be appreciated that the compounds of formula (I) can have metal binding, chelating, complex forming properties and therefore can exist as metal complexes or metal chelates. Such metalated derivatives of the compounds of formula (I) are intended to be included within the scope of the present invention.

Some of the compounds of formula (I) can also exist in their tautomeric form. Such forms although not explicitly indicated in the above formula are intended to be included within the scope of the present invention.

The term “alkyl” as used herein it does refer to any linear, branched, or cyclic hydrocarbon in which all carbon-carbon bonds are single bonds.

The term “alkenyl” and “unsubstituted alkenyl” are used interchangeably herein and refer to any linear, branched, or cyclic alkyl with at least one carbon-carbon double bond.

Furthermore, the term “alkynyl” as used herein it does refer to any linear, branched, or cyclic alkyl or alkenyl with at least one carbon-carbon triple bond.

The term “aryl” as used herein it does refer to any aromatic cyclic alkenyl or alkynyl, being as a group or part of a group is phenyl or naphthalenyl, each optionally substituted with one, two or three substituents selected from halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or diC₁₋₆alkylamino, azido, mercapto, polyhaloC₁₋₆alkyl, and polyhaloC₁₋₆alkoxy. The term “alkaryl” is employed where an aryl is covalently bound to an alkyl, alkenyl, or alkynyl.

The term “substituted” as used herein refers to a replacement of an atom or chemical group (e.g., H, NH₂, or OH) with a functional group, and particularly contemplated functional groups include nucleophilic groups (e.g., —NH₂, —OH, —SH, —NC, etc.), electrophilic groups (e.g., C(O)OR, C(X)OH, etc.), polar groups (e.g., —OH), non-polar groups (e.g., aryl, alkyl, alkenyl, alkynyl, etc.), ionic groups (e.g., —NH₃⁺), and halogens (e.g., —F, —Cl), and all chemically reasonable combinations thereof. Thus, the term “functional group” and the term “substituent” are used interchangeably herein and refer to nucleophilic groups (e.g., —NH₂, —OH, —SH, —NC, —CN, etc.), electrophilic groups (e.g., C(O)OR, C(X)OH, C(Halogen)OR, etc.), polar groups (e.g., —OH), non-polar groups (e.g., aryl, alkyl, alkenyl, alkynyl, etc.), ionic groups (e.g., —NH₃⁺), and halogens.

For the purposes of present description, the term “Nucleoside Phosphorylase” or “NP” or “NPs” comprises Pyrimidine Nucleoside Phosphorylase enzymes or PyNP (PyNPs) enzymes or mixtures of PyNPs and PNPs (Purine Nucleoside Phosphorylase enzymes). Those enzymes can be obtained from naturally occurring microorganisms, either mesophiles, thermophiles, hyperthermophiles or extremophiles. For the purpose of the present description organisms or NPs enzymes, mesophiles or mesophilic are those able to work or to carry out a NP activity, at temperatures ranging from 18 to 60° C., with an optimal temperature range of 40-55° C. Organisms or NPs enzymes, thermophiles or thermophilic are those able to work or to carry out a NP activity, at temperatures over 60° C. and up to 80° C. Organisms or NPs enzymes, hyperthermophiles or hyperthermophilic are those able to work or to carry out a NP activity, at temperatures over 80° C. an up to 100° C. with an optimal temperature range of 80-95° C. Additionally, the enzymes used in present invention can be cloned enzymes, obtained by genetic recombination techniques, expressed in host cells transformed with the corresponding vectors carrying the respective encoding nucleic acid sequences.

The nucleic acid molecule encoding a NP enzyme according to present invention is preferably selected from: SEQ ID NO. 1, 2, 5, 7, 9 or 11; or
a) a nucleotide sequence which is the complement of SEQ ID. NO:1, 2, 5, 7, 9 or 11; or
b) a nucleotide sequence which is degenerate with SEQ ID. NO: 1, 2, 5, 7, 9 or 11; or
c) a nucleotide sequence hybridizing under conditions of high stringency to SEQ ID. NO:1, 2, 5, 7, 9 or 11; to the complement of SEQ ID. NO:1, 2, 5, 7, 9 or 11; or to a hybridization probe derived from SEQ ID. NO: 1, 2, 5, 7, 9 or 11; or their complement thereof; or
d) a nucleotide sequence having at least 80% sequence identity with SEQ ID. NO: 1, 2, 5, 7, 9 or 11; or
e) a nucleotide sequence having at least 59% sequence identity with SEQ ID. NO: 1, 2, 5, 7, 9 or 11; or
f) a nucleotide sequence encoding for an amino acid sequence selected from: SEQ ID. NO: 3, 4, 6, 8, 10 or 12,

Conditions of stringency hybridization in the sense of the present invention are defined as those described by Sambrook et al., Molecular Cloning, A Laboratory Manual, Cold Spring Harbor Laboratory Press (1989), 1.1011.104. According to this, hybridization under stringent conditions means that a positive hybridization signal is still observed after washing for one hour with 1×SSC buffer and 0.1% SDS at 55° C. In some aspects, the wash is conducted at 62° C. In other aspects, washes are conducted at 68° C. In some particular aspects, the wash is conducted for one hour in 0.2×SSC buffer and 0.1% SDS at 55° C.

Moreover, in the sense of present description, the invention also covers nucleotide or amino acid sequences which, on nucleotide or amino acidic levels, respectively, have an identity of at least 59%, at least 80%, or at least 90% to the nucleotide or amino acid sequence shown in SEQ ID NO. 1 or SEQ ID NO: 2 (nucleotidic) or SEQ ID NO. 3 or SEQ ID NO:4, (amino acidic). Percent identity are determined according to the following equation:

I=(n/L)×100

wherein I are percent identity, L is the length of the basic sequence and n is the number of nucleotide or amino acid difference of a sequence to the basic sequence.

TABLE 1
Identity of purine nucleoside phosporylase (deoD) [Sulfolobus
solfataricus] protein sequences with similar PNP proteins in GenBank
(www.ncbi.nlm.nih.gov/genbank/, accessed on 15 Nov. 2013).

| Accession number | Organism | Identity % |
|---|---|---|
| NP_342951.1 | Sulfolobus solfataricus P2 | 100% |
| | (SEQ ID NO: 3) | |
| NP_378450.1 | Sulfolobus tokodaii str. 7 | 83% |
| YP_005648757.1 | Sulfolobus islandicus LAL14/1 | 59% |
| WP_016730864.1 | Sulfolobus islandicus | 64% |
| WP_016730167.1 | Sulfolobus islandicus | 61% |

TABLE 2
Identity of uridine phosphorylase [Aeropyrum pernix
K1 protein sequences with similar UDP proteins in GenBank
(www.ncbi.nlm.nih.gov/genbank/, accessed on 15 Nov. 2013).

| Accession number | Organism | Identity % |
|---|---|---|
| NP_148386.2 | Aeropyrum pernix K1 (SEQ ID NO: 4) | 100% |
| YP_008604720.1 | Aeropyrum camini SY1 | 93% |

Still another subject matter of the present invention is a recombinant vector comprising at least one copy of the nucleic acid molecules as defined above, operatively linked with an expression control sequence. The vector can be any prokaryotic or eukaryotic vector. Examples of prokaryotic vectors are chromosomal vectors such as bacteriophages (e. g. bacteriophage Lambda) and extrachromosomal vectors such as plasmids (see, for example, Sambrook et al., supra, Chapter 1-4). The vector can also be a eukaryotic vector, e. g. a yeast vector or a vector suitable for higher cells, e. g. a plasmid vector, viral vector or plant vector. Suitable eukaryotic vectors are described, for example, by Sambrook et al., supra, Chapter 16. The invention moreover relates to a recombinant cell transformed with the nucleic acid or the recombinant vector as described above. The cell can be any cell, e. g. a prokaryotic or eukaryotic cell. In some aspects, the cells are prokaryotic cells. In some aspects, the cells are E. coli cells.

For the purpose of present description, the invention also covers variants, or orthologues of SEQ ID NO: 1 to SEQ ID NO:12.

The term “variant” as used throughout the specification is to be understood to mean a nucleotide sequence of a nucleic acid or amino acid sequence of a protein or polypeptide that is altered by one or more nucleotides or amino acids, respectively. The variant can have “conservative” changes, wherein a substituted nucleotide or amino acid has similar structural or chemical properties to the replaced nucleotide or amino acid. A variant can also have “non-conservative” changes or a deletion and/or insertion of one or more nucleotides or amino acids. The term also includes within its scope any insertions/deletions of nucleotides or amino acids for a particular nucleic acid or protein or polypeptide. A “functional variant” will be understood to mean a variant that retains the functional capacity of a reference nucleotide sequence or a protein or polypeptide.

The term “complement” or “complementary” as used herein means, for example, that each strand of 20 double-stranded nucleic acids such as, DNA and RNA, is complementary to the other in that the base pairs between them are non-covalently connected via two or three hydrogen bonds. For DNA, adenine (A) bases complement thymine (T) bases and vice versa; guanine (G) bases complement cytosine (C) bases and vice versa. With RNA, it is the same except that adenine (A) bases complement uracil (U) bases instead of thymine (T) bases. Since there is only one 25 complementary base for each of the bases found in DNA and in RNA, one can reconstruct a complementary strand for any single strand.

The term “orthologue” as used throughout the specification is to be understood as homologous gene or miRNAs sequences found in different species.

A “recombinant DNA molecule” is understood under present specification, as a DNA molecule that has undergone a molecular biological manipulation. The term “recombinant DNA” therefore, it is a form of DNA that does not exist naturally, which is created by combining DNA sequences that would not normally occur together.

Once introduced into a host cell, a “recombinant DNA molecule” is replicated by the host cell. By “recombinant protein or enzyme” herein is meant a protein or enzyme produced by a method employing a “recombinant DNA molecule” and hence, also for the purposes of present description, the term recombinant enzyme or recombinant type enzyme should be understood as a protein or enzyme that is derived from recombinant DNA.

“Mutant” enzyme, for the purposes of present specification is meant by any enzyme which shows at least one mutation, either naturally occurring of induced by human manipulation, including genetic engineering.

On the contrary, native, natural or naturally occurring enzyme, for the purposes of present specification all taken as synonyms, is meant by an enzyme which conserves its former amino acid sequence as it is found widespread mostly in nature, without mutations.

By “functional part” or “functional fragment” of the enzymes of present invention is meant any sequence fragment of the original enzyme, or of a DNA segment encoding the same, that keeps the ability of the full enzyme sequence to carry out all its biocatalysis properties.

The term cytosinic nucleoside analogues means, for the purposes of present specification, either nucleoside analogues of cytosine or nucleoside analogues of cytosine derivatives. Equally, the term cytosinic base means, as constrained to the present patent specification, the base cytosine or derivatives thereof. Analogously, the term modified cytosinic base, as used in present specification, covers compounds represented by formula II, wherein, the skeleton of cytosine base is substituted in positions 4, 5 or 6.

Present description discloses a enzymatic process for producing cytosinic nucleoside analogues, particularly useful as active pharmaceutical ingredients (APIs), intermediates or prodrugs thereof, being those APIs or their intermediates, nucleoside analogues (NAs) of formula I:

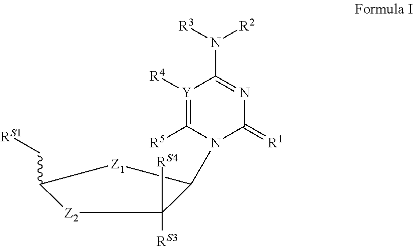

wherein,
Z₁ being: O, CH₂, S, NH;
Z₂ being, independently of Z₁: O, C(R^{S2}R^{S5}), S(R^{S2}R^{S5}), S(R^{S2}), S(R^{S5}), preferably, a group SO or SO₂; N(R^{S2}R^{S5}), N(R^{S2}), N(R^{S5});
R^{S1} being: hydrogen, OH, ether or ester thereof selected from:

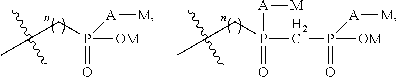

being n is 0 or 1, A is oxygen or nitrogen, and each M is independently hydrogen, an optionally substituted alkyl chain, an optionally substituted alkenyl chain, an optionally substituted alkynyl chain, an optionally substituted aryl optionally linked to P by an optionally substituted alkyl, alkenyl or alkynyl chain, an optionally substituted heterocycle optionally linked to P by an optionally substituted alkyl, alkenyl or alkynyl chain, or a pharmaceutically acceptable counter-ion such as, but not restricted to, sodium, potassium, ammonium or alkylammonium;
R^{S2} being hydrogen, OH or an ether or ester residue thereof, halogen, preferably F, CN, NH₂, SH, C≡CH, N₃;
R^{S3} being hydrogen, in case of NA derived from 2′-deoxyribonucleosides or arabinonucleosides or being selected from: OH, NH₂, halogen, preferably F, OCH₃, when the NA is derived from ribonucleosides;
R^{S4} being hydrogen, OH or an ether or ester residue thereof, NH₂, halogen, preferably F, CN;
providing R^{S1} and R^{S4} are different when both were ethers or esters of OH residues;
R^{S5} being hydrogen, OH or an ether or ester residue thereof, NH₂ or halogen, preferably F, when Z₂ is different from oxygen;
R¹ being O, CH₂, S, NH;
R² being hydrogen, an optionally substituted alkyl chain, preferably C₄₋₄₀ alkyl chain, an optionally substituted alkenyl chain, an optionally substituted alkynyl chain, an optionally substituted aryl linked to N by an optionally substituted alkyl, alkenyl or alkynyl chain, an optionally substituted heterocycle linked to N by an optionally substituted alkyl, alkenyl or alkynyl chain, COR⁶, CONR⁶R⁷, CO₂R⁶, C(S)OR⁷, CN, SR⁶, SO₂R⁶, SO₂R⁶R⁷, CN, P(O)aryl, P(O)heterocycle, P(S)aryl, P(S)heterocycle, P(O)O₂R⁸;
R³ being hydrogen, an optionally substituted alkyl chain, preferably C₄₋₄₀ alkyl chain, an optionally substituted alkenyl chain, an optionally substituted alkynyl chain, an optionally substituted aryl linked to N by an optionally substituted alkyl, alkenyl or alkynyl chain, an optionally substituted heterocycle linked to N by an optionally substituted alkyl, alkenyl or alkynyl chain, COR⁶, CONR⁶R⁷, CO₂R⁶, C(S)OR⁷, CN, SR⁶, SO₂R⁶; SO₂R⁶R⁷, CN, P(O)aryl, P(O)heterocycle, P(S)aryl, P(S)heterocycle, P(O)O₂R⁸; being R¹ and R² independent one from each other; and providing that at least one R₂ or R₃ is different from hydrogen.
R⁴ being hydrogen, OH, NH₂, SH, halogen (preferably F or I); optionally substituted alkyl chain; optionally substituted alkenyl chain; optionally substituted alkynyl chain, trihaloalkyl, OR⁶, NR⁶R⁷, CN, COR⁶, CONR⁶R⁷, CO₂R⁶, C(S)OR⁶, OCONR⁶R⁷, OCO₂R⁶, OC(S)OR⁶, NHCONR⁶R⁷, NHCO₂R⁶, NHC(S)OR⁶, SO₂NR⁶R⁷, an optionally substituted aryl linked to Y by an optionally substituted alkyl, alkenyl or alkynyl chain, an optionally substituted heterocycle linked to Y by an optionally substituted alkyl, alkenyl or alkynyl chain, and any optionally substituted heterocycle or optionally substituted aryl of, independently, R¹, R², R³, R⁴ or R⁵, selected from:

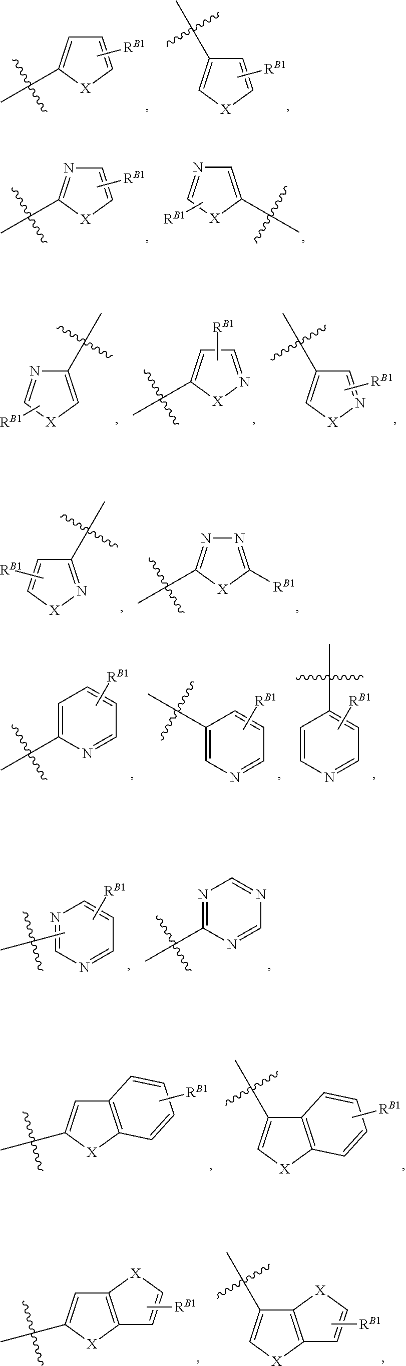

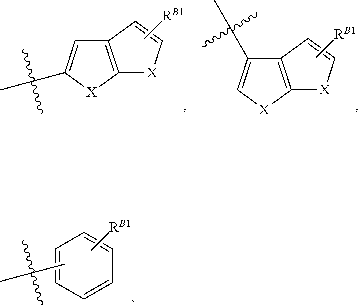

providing Y is a carbon or sulphur atom and, alternatively, R⁴ being absent, providing Y is a nitrogen atom;
wherein
X being O, S, N—R^{B2}, Se; R^{B1} being H, OH, NH₂, SH, straight or branched C₁₋₁₀ alkyl, F, Cl, Br, I, X—R^{B2}, —C≡C—R^{B2}, CO₂R^{B2}; R^{B2} being H, OH, NH₂, straight or branched C₁₋₅ alkyl, phenyl;
R⁵ being hydrogen, OH, NH₂, SH, halogen (preferably F or I), an optionally substituted alkyl chain, an optionally substituted alkenyl chain, an optionally substituted alkynyl chain, trihaloalkyl, OR⁶, NR⁶R⁷, CN, COR⁶, CONR⁶R⁷, CO₂R⁶, C(S)OR⁶, OCONR⁶R⁷, OCO₂R⁶, OC(S)OR⁶, NHCONR⁶R⁷, NHCO₂R⁶, NHC(S)OR⁶, SO₂NR⁶R⁷; CH₂-heterocyclic ring, CN;
and any optionally substituted heterocycle or optionally substituted aryl of, independently, R¹, R², R³, R⁴ or R⁵, selected from:

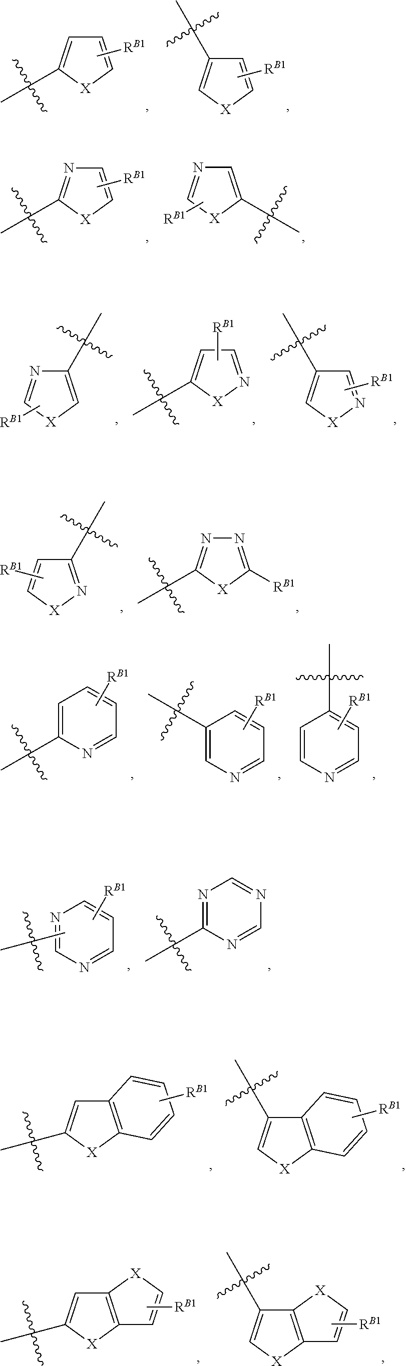

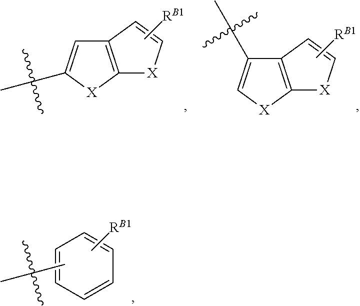

wherein
X being O, S, N—R², S; R^{B1} being H, OH, NH₂, SH, straight or branched C₁₋₁₀ alkyl, F, Cl, Br, I, X—R^{B2}, —C≡C—R^{B2}, CO₂R^{B2}; R^{B2} being H, OH, NH₂, straight or branched C₁₋₅ alkyl, phenyl;
R⁶ and R⁷ are independently of each other hydrogen, optionally substituted alkyl chain, optionally substituted alkenyl chain, optionally substituted alkynyl chain, heterocyclic or optionally substituted aryl;
R⁸ being hydrogen, an optionally substituted alkyl chain, an optionally substituted alkenyl chain, an optionally substituted alkynyl chain, an optionally substituted aryl an optionally substituted heterocycle,
Y being C, N, S;
wherein the process for producing the cytosinic nucleoside analog comprises the following steps, when a chemo-enzymatic embodiment of the process of the invention is employed:
(i) chemically reacting the cytosinic base with suitable reagents for modifying the amino group at N⁴ position in cytosine derivative in order to incorporate the proper substitution described as substituent R² and R³ in intermediate compound of formula II which is optionally purified at the end of step (i) by conventional purification methods;
   alternatively, the process of invention can depart directly from the starting products represented by formula II (modified cytosinic base), either available in the market or previously synthesized

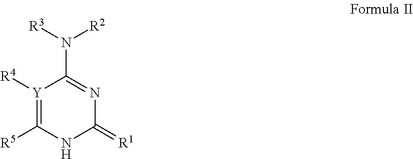

(ii) reacting the above mentioned modified cytosinic base with a suitable nucleoside analog starting material, wherein said reaction comprises the addition, in a suitable reaction aqueous medium and under suitable reaction conditions, of a nucleoside phosphorylase enzyme (NPs), preferably a Pyrimidine Nucleoside Phosphorylase enzyme (PyNP), to a mixture of starting materials comprising a cytosine derivative of formula II and a nucleoside analog of formula III

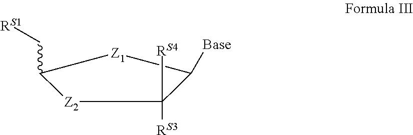

wherein,
Z₁ being O, CH₂, S, NH;
Z₂ being, independently of Z₁: O, C(R^{S2}R^{S5}), S(R^{S2}R^{S5}), S(R^{S2}), S(R^{S5}), preferably, a group SO or SO₂; N(R^{S2}R^{S5}), N(R^{S2}), N(R^{S5});
R^{S1} being hydro en, OH, ether or ester thereof selected from:

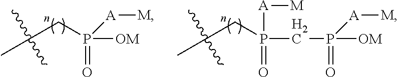

being n is 0 or 1, A is oxygen or nitrogen, and each M is independently hydrogen, an optionally substituted alkyl chain, an optionally substituted alkenyl chain, an optionally substituted alkynyl chain, an optionally substituted aryl optionally linked to P by an optionally substituted alkyl, alkenyl or alkynyl chain, an optionally substituted heterocycle optionally linked to P by an optionally substituted alkyl, alkenyl or alkynyl chain, or a pharmaceutically acceptable counter-ion such as, but not restricted to, sodium, potassium, ammonium or alkylammonium;
R^{S2} being hydrogen, OH or an ether or ester residue thereof, halogen, preferably F, CN, NH₂, SH, C≡CH, N₃;
R^{S3} being hydrogen, in case of NA derived from 2′-deoxyribonucleosides or arabinonucleosides or being selected from: OH, NH₂, halogen (preferably F), OCH₃, when the NA is derived from ribonucleosides;
R^{S4} being hydrogen, OH or an ether or ester residue thereof, NH₂, halogen, preferably F, CN;
providing R^{S1} and R^{S4} are different when both were ethers or esters of OH residues;

R^{S5} being hydrogen, OH or an ether or ester residue thereof, NH₂ or halogen, preferably F, in the case that Z₂ is different from oxygen;
R¹ being O, CH₂, S, NH;
R² being hydrogen, an optionally substituted alkyl chain, preferably C₄₋₄₀alkyl chain, an optionally substituted alkenyl chain, an optionally substituted alkynyl chain, an optionally substituted aryl linked to N by an optionally substituted alkyl, alkenyl or alkynyl chain, an optionally substituted heterocycle linked to N by an optionally substituted alkyl, alkenyl or alkynyl chain, COR⁶, CONR⁶R⁷, CO₂R⁶, C(S)OR⁷, CN, SR⁶, SO₂R⁶, SO₂R⁶R⁷, CN, P(O)aryl, P(O)heterocycle, P(S)aryl, P(S)heterocycle, P(O)O₂R⁸;
R³ being hydrogen, an optionally substituted alkyl chain, preferably C₄₋₄₀alkyl chain, an optionally substituted alkenyl chain, an optionally substituted alkynyl chain, an optionally substituted aryl linked to N by an optionally substituted alkyl, alkenyl or alkynyl chain, an optionally substituted heterocycle linked to N by an optionally substituted alkyl, alkenyl or alkynyl chain, COR⁶, CONR⁶R⁷, CO₂R⁶, C(S)OR⁷, CN, SR⁶, SO₂R⁶; SO₂R⁶R⁷, CN, P(O)aryl, P(O)heterocycle, P(S)aryl, P(S)heterocycle, P(O)O₂R⁸; being R³ and R² independent one from each other; and providing that at least one R₂ or R₃ is different from hydrogen;
R⁴ being hydrogen, OH, NH₂, SH, halogen (preferably F or I); optionally substituted alkyl chain; optionally substituted alkenyl chain; optionally substituted alkynyl chain, trihaloalkyl, OR⁶, NR⁶R⁷, CN, COR⁶, CONR⁶R⁷, CO₂R⁶, C(S)OR⁶, OCONR⁶R⁷, OCO₂R⁶, OC(S)OR⁶, NHCONR⁶R⁷, NHCO₂R⁶, NHC(S)OR⁶, SO₂NR⁶R⁷, an optionally substituted aryl linked to Y by an optionally substituted alkyl, alkenyl or alkynyl chain, an optionally substituted heterocycle linked to Y by an optionally substituted alkyl, alkenyl or alkynyl chain, and any optionally substituted heterocycle or optionally substituted aryl of, independently, R², R³, R⁴ or R⁵, selected from:

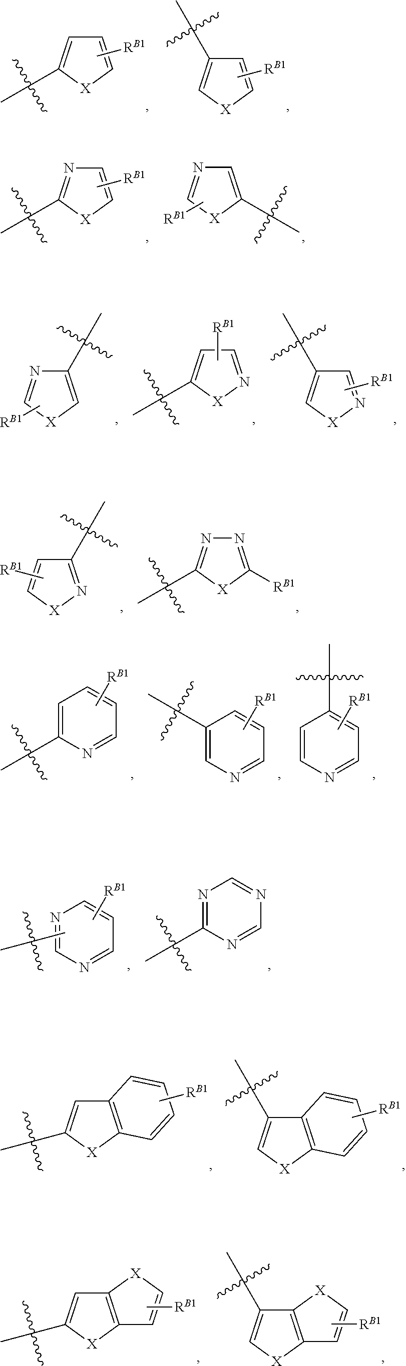

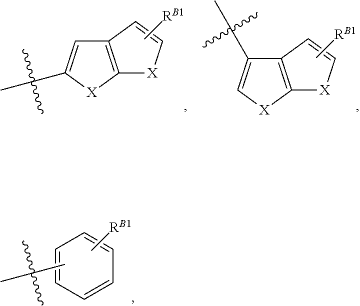

providing Y is a carbon or sulphur atom and, alternatively, R⁴ being absent, providing Y is a nitrogen atom;
wherein
X being O, S, R^{B2}, Se; R^{B1} being H, OH, NH₂, SH, straight or branched C₁₋₁₀ alkyl, F, Cl, Br, I, X—R¹², —C≡C—R², CO₂R^{B2}; R^{B2} being H, OH, NH₂, straight or branched C₁₋₅ alkyl, phenyl;
R⁵ being hydrogen, OH, NH₂, SH, halogen (preferably F or I), an optionally substituted alkyl chain, an optionally substituted alkenyl chain, an optionally substituted alkynyl chain, trihaloalkyl, OR⁶, NR⁶R⁷, CN, COR⁶, CONR⁶R⁷, CO₂R⁶, C(S)OR⁶, OCONR⁶R⁷, OCO₂R⁶, OC(S)OR⁶, NHCONR⁶R⁷, NHCO₂R⁶, NHC(S)OR⁶, SO₂NR⁶R⁷; CH₂-heterocyclic ring, CN;
and any optionally substituted heterocycle or optionally substituted aryl of, independently, R², R³, R⁴ or R⁵, selected from:

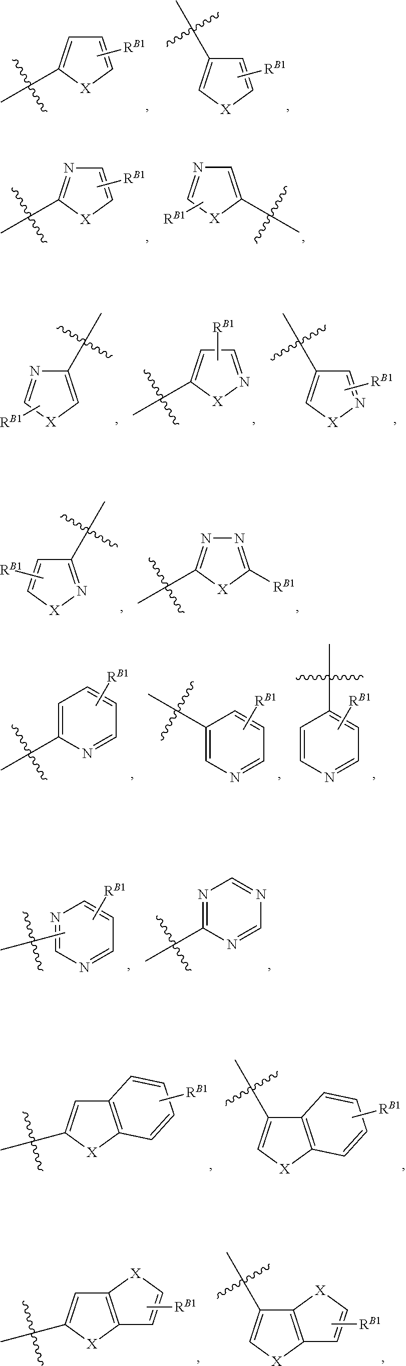

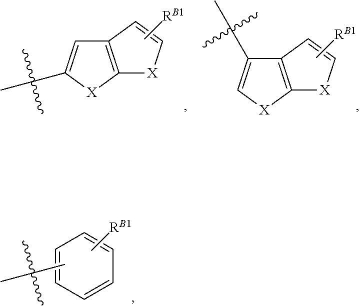

wherein
X being O, S, N—R^{B2}, Se; R^{B1} being H, OH, NH₂, SH, straight or branched C₁₋₁₀ alkyl, F, Cl, Br, I, X—R^{B2}, —C≡C—R^{B2}, CO₂R^{B2}; R^{B2} being H, OH, NH₂, straight or branched C₁₋₁₀ alkyl, phenyl;
R⁶ and R⁷ are independently of each other hydrogen, optionally substituted alkyl chain, optionally substituted alkenyl chain, optionally substituted alkynyl chain, heterocyclic or optionally substituted aryl;
R⁸ being hydrogen, an optionally substituted alkyl chain, an optionally substituted alkenyl chain, an optionally substituted alkynyl chain, an optionally substituted aryl an optionally substituted heterocycle;
Y being C, N, S;
(iii) optionally, deprotecting the amino group at N⁴ position in cytosinic nucleoside analogue in order to recover the free primary amino N⁴ at the cytosinic nucleoside analogue of formula I which was further purified by conventional purification methods.

Preferably the heterocyclic ring constituting the base in formula III of the starting materials is selected from: uracil, adenine, cytosine, guanine, thymine, hypoxanthine, xanthine, thiouracil, thioguanine, 9-H-purine-2-amine, 7-methylguanine, 5-fluorouracil, 5-bromouracil, 5-chlorouracil, 5,6-dihydrouracil, 5-methylcytosine and 5-hydroxymethylcytosine and any substituted derivatives thereof.

Moreover, the free cytosine or cytosine derivative to be modified in order to obtain the corresponding nucleobase of formula IT is preferably selected from:

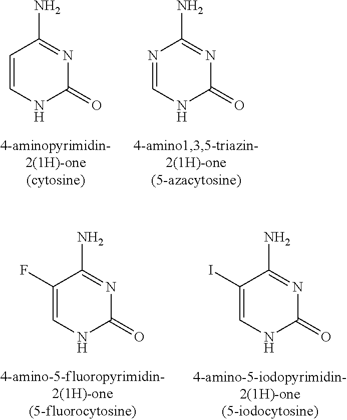 [SMILES: Nc1cc[nH]c(=O)n1 ; Nc1nc[nH]c(=O)n1 ; Nc1nc(=O)[nH]cc1F ; Nc1nc(=O)[nH]cc1I]

Moreover, the cytosinic nucleobase in the nucleoside analog of formula I is preferably selected from:

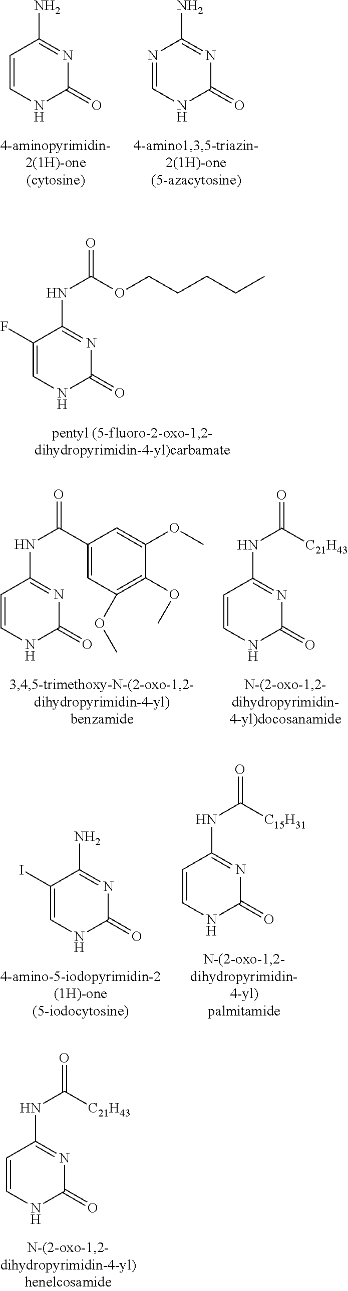

With the process described herein, the APIs, intermediates or prodrugs thereof produced are selected from: Capecitabine, Decitabine (aza-dCyd or DAC), 5-Azacytidine (aza-Cyd), Cytarabine (ara-C), Enocitabine (BH-AC), Gemcitabine (dFdC), Zalcitabine (ddC), Ibacitabine, Sapacitabine, 2′-C-cyano-2′-deoxy-1-β-D-arabino-pentofuranosylcytosine (CNDAC), Galocitabine, Valopricitabine (NM283), 2′-Deoxy-4′-thiocytidine, Thiarabine (T-araC), 2′-Deoxy-4′-thio-5-azacytidine or Apricitarabine (ATC).

More preferably, the APIs, intermediates or prodrugs thereof produced are selected from: Capecitabine, Decitabine (aza-dCyd or DAC), 5-Azacytidine (aza-Cyd), Cytarabine (ara-C), and still more preferably the described process is particularly intended to the industrial production of Capecitabine or Cytarabine.

Still in one more embodiment of practicing the process describe hereto, the API, intermediates or prodrugs thereof, produced is Capecitabine (Scheme 2).

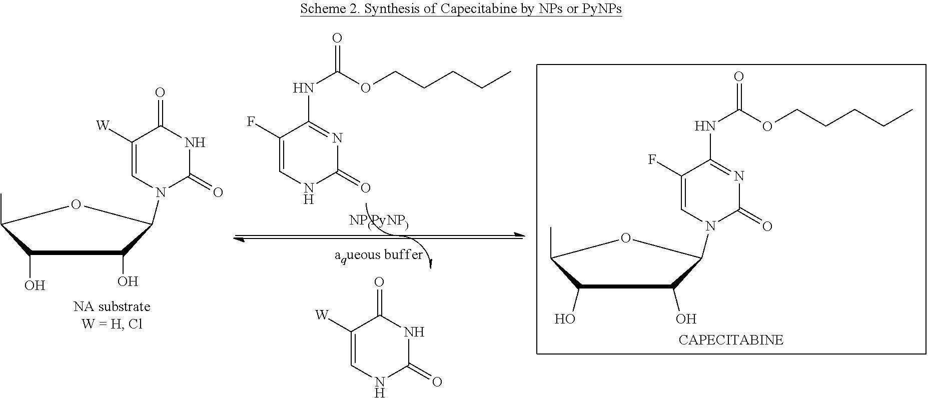

Biocatalytic steps (enzymatic steps) of Capecitabine either enzymatic or chemo-enzymatic synthesis were carried out at temperatures preferably ranging 20-120° C.

Still in one more embodiment of practicing the process described hereto, the API, intermediates or prodrugs thereof, produced is Cytarabine (Scheme 3).

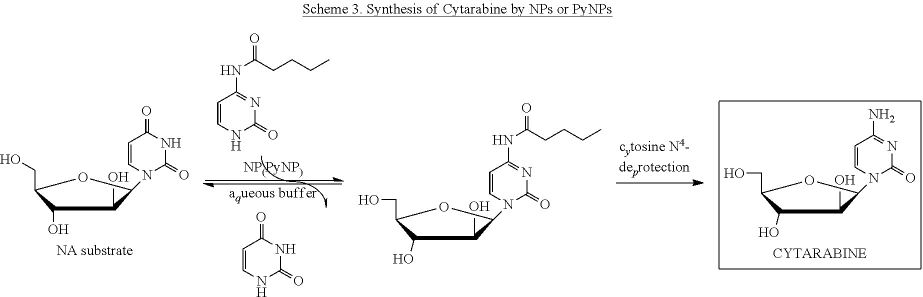

For the purposes of carrying out the biocatalytic process of present description, the applicant has chosen organisms containing NPs enzymes or NPs enzymes isolated as such, preferably, in both cases, PyNP enzymes or PyNP/PNP mixtures of enzymes, wherein either the enzymes or the microorganisms containing them, were mesophiles or mesophilic, in the sense that they were able to work and to carry out a nucleoside transferase activity, at temperatures ranging from 18 to up 60° C., with an optimal temperature range of 40-55° C. Organisms or NPs enzymes, particularly PyNP enzymes, thermophiles or thermophilic are those able to work or to carry out a nucleoside transferase activity, at temperatures ranging over 60° C. and up to 80° C. Organisms or NPs enzymes, particularly PyNP enzymes, hyperthermophiles or hyperthermophilic, are those able to work or to carry out a nucleoside transferase activity, at temperatures over 80° C. and up to 100° C., with an optimal temperature range of 80-95° C.

The NPs (PyNP) enzyme used in the process of invention can be isolated from a microorganism selected from mesophilic organisms, as a way of example, bacteria, particularly from E. coli or from mesophilic, thermophilic or hyperthermophilic Archaea particularly from Thermoprotei class and more particularly selected from the genus and species disclosed in WO2011/076894. In some aspects, nucleoside phosphorylases, according to present invention are from Sulfolobus solfataricus and Aeropyrum pernix.

Still another embodiment of present invention relates to the use in the process disclosed of a recombinant NP enzyme comprising an amino acid sequence encoded by a nucleic acid sequence, or fragments thereof, selected from SEQ ID NO: 1 or SEQ ID NO: 2. Both DNA sequences are isolated from Archaea and, more particularly, from Archaea having homologous sequences to nucleoside phosphorylase enzymes.

In one embodiment the process disclosed herein is based in the use as biocatalyst of mesophilic, thermophilic or hyperthermophilic NPs enzymes, either Purine NPs (PNPs), or Pyrimidine NPs (PyNPs), or mistures thereof, for performing the base transfer one step-one pot reaction. Preferably thermophilic or hyperthermophilic NPs enzymes are of recombinant type, comprising gene sequences of fragments thereof, encoding for NP enzymatic activities able to carry out nucleobase transfer one step-one pot reactions, at temperatures ranging over 60° C. and up to 100° C. and, more preferably, at the suitable reaction conditions described herein.

For the purposes of present description, some enzymes suitable to be used in the biocatalytic process of invention, methods of cloning the same, vectors carrying the nucleic acid sequences encoding the same and host cells transformed with the aforesaid vectors, are disclosed in WO2011/076894 (see also, U.S. Pat. No. 8,512,997. U.S. Pat. No. 8,759,034), in the name of some of the inventors of present invention and whose patent application whole specification is enclosed herein in full, by reference.

Suitable conditions for carrying out the different embodiments of the process of invention comprise:
a) Temperature ranging 20-120° C.
b) Reaction time ranging 1-1000 h
c) Concentration of starting material ranging 1-1000 mM
d) Stoichiometry nucleoside starting material:nucleobase ranges from 1:5 to 5:1.
e) Amount of NP enzyme ranging 0.001-100 mg/ml
f) Nucleobase added to the reaction medium, optionally dissolved in an organic solvent
g) Aqueous reaction medium optionally also containing up to 40% of a suitable organic solvent. Preferably up to 20% and more preferably up to 5%.

Optionally, organic solvents could be added to the reaction medium or be used for dissolving previously the nucleobases. In some aspects, the solvents are polar aprotic solvents. In some aspects, the solvents are, for example, tetrahydrofuran, acetonitrile, acetone, dimethylformamide (DMF), dimethylsulfoxide (DMSO), and combinations thereof.

The process according to present invention can also comprise isolation and/or purification steps of the NA produced by standard operation means selected from: precipitation, filtration, concentration or crystallization.

Thermophilic NP enzymes are able to operate at higher temperatures which render the nucleobase transfer reaction more efficient in terms of time and overall yield.

The process of the invention, specifically disclosed as embodiment of the invention, the production of Capecitabine (Scheme 2), wherein the ribonucleoside used as starting material is 5′-deoxyribofuranosyl nucleoside selected from: 5′-deoxyuridine, 5′-deoxy-5-methyluridine or 5′-chloro-5′-deoxyuridine; the nucleobase used as starting material to be transferred by the PyNP enzyme, is pentyl (5-fluoro-2-oxo-1,2-dihydropyrimidin-4-yl)carbamate and the PyNP is a naturally occurring mesophilic, thermophilic or hyperthermophilic enzyme, isolated from bacteria.

One alternative embodiment of the process of invention is the one wherein the API produced is, Capecitabine (Scheme 2), the ribonucleoside used as starting material is 5′-deoxyuridine, 5′-deoxy-5-methyluridine or 5′-chloro-5′-deoxyuridine, the nucleobase also used as starting material to be transferred by the PyNP enzyme, is pentyl (5-fluoro-2-oxo-1,2-dihydropyrimidin-4-yl)carbamate and the PyNP is a recombinant mesophilic, thermophilic or hyperthermophilic enzyme, whose cloned DNA was isolated from bacteria.

A further embodiment of the process of invention is the one wherein the API produced is, Capecitabine (Scheme 2), the ribonucleoside used as starting material is 5′-deoxyuridine, 5′-deoxy-5-methyluridine or 5′-chloro-5′-deoxyuridine, the nucleobase also used as starting material to be transferred by the PyNP enzyme, is pentyl (5-fluoro-2-oxo-1,2-dihydropyrimidin-4-yl)carbamate and the PyNP is a naturally occurring mesophilic, thermophilic or hyperthermophilic enzyme, isolated from Archaea.

Still another embodiment of the process of invention is the one wherein the API produced is, Capecitabine (Scheme 2), the ribonucleoside used as starting material is 5′-deoxyuridine, 5′-deoxy-5-methyluridine or 5′-chloro-5′-deoxyuridine, the nucleobase also used as starting material to be transferred by the PyNP enzyme, is pentyl (5-fluoro-2-oxo-1,2-dihydropyrimidin-4-yl)carbamate and the PyNP is a recombinant mesophilic, thermophilic or hyperthermophilic enzyme, isolated from Archaea.

The process of the invention, also is represented by the one wherein the API produced is Cytarabine (Scheme 3), the ribonucleoside used as starting material is arabinofuranosyluracil or arabinonucleosides, the nucleobase also used as starting material to be transferred by the PyNP enzyme, is N-(2-oxo-1,2-dihydropyrimidin-4-yl)pentanamide and the PyNP is a naturally occurring mesophilic, thermophilic or hyperthermophilic enzyme, isolated from bacteria.

Another one of the embodiments of the process of invention is that wherein the API produced is Cytarabine (Scheme 3), the ribonucleoside used as starting material is arabinofuranosyluracil or arabinonucleosides, the nucleobase also used as starting material to be transferred by the PyNP enzyme, is N-(2-oxo-1,2-dihydropyrimidin-4-yl)pentanamide and the PyNP is a recombinant mesophilic, thermophilic or hyperthermophilic enzyme, whose cloned DNA was isolated from bacteria.

An additional embodiments of the process of invention is the one wherein the API produced is, Cytarabine (Scheme 3), the ribonucleoside used as starting material is arabinofuranosyluracil or arabinonucleosides, the nucleobase also used as starting material to be transferred by the PyNP enzyme, N-(2-oxo-1,2-dihydropyrimidin-4-yl)pentanamide and the PyNP is a naturally occurring mesophilic, thermophilic or hyperthermophilic enzyme, whose cloned DNA was isolated from Archaea.

Another embodiments of the process of invention is the one wherein the API produced is, Cytarabine (Scheme 3), the ribonucleoside used as starting material is arabinofuranosyluracil or arabinonucleosides, the nucleobase also used as starting material to be transferred by the PyNP enzyme, N-(2-oxo-1,2-dihydropyrimidin-4-yl)pentanamide and the PyNP is a recombinant mesophilic, thermophilic or hyperthermophilic enzyme, whose cloned DNA was isolated from Archaea.

Forming part of the same inventive concept, the present description also discloses recombinant nucleoside phosphorylase enzymes (either PNPs or PyNPs), for use in any of the processes of the invention as described above. The said native or recombinant NP enzymes can be isolated from mesophilic organisms more preferably, bacteria and Archaea; or from thermophilic organisms more preferably, bacteria and Archaea; or hyperthermophilic organisms, more preferably bacteria and Archaea, and more particularly from Sulfolobus solfataricus and Aeropyrum pernix.

Also in the same line of integrating a single inventive linked concept, present description also discloses the use of a mesophilic, thermophilic or hyperthermophilic nucleoside phosphorylase (NP o PyNP) in the production of APIs, intermediates or prodrugs thereof, being those APIs, their intermediates or their prodrugs, cytosine nucleoside analogues (NAs) useful as anti-cancer or anti-viral medicaments. More preferably, the previously mentioned use is achieved by the production process and variants thereof, also previously detailed, of NAs as APIs, intermediates or prodrugs thereof. Particularly, related to that previously mentioned use, recombinant thermophilic nucleoside phosphorylase (PNPs, PyNPs or mixtures thereof) are preferred, in the production of APIs, being those APIs or their intermediates, nucleoside analogues (NAs) particularly useful as anti-cancer or anti-viral medicaments.

Among the APIs, intermediates or prodrugs thereof produced by such uses the following can be found:
Capecitabine, Decitabine (aza-dCyd or DAC), 5-Azacytidine (aza-Cyd), Cytarabine (ara-C), Enocitabine (BH-AC), Gemcitabine (dFdC), Zalcitabine (ddC), Ibacitabine, Sapacitabine, 2′-C-cyano-2′-deoxy-1-β-D-arabino-pentofuranosylcytosine (CNDAC), Galocitabine, Valopricitabine (NM283), 2′-Deoxy-4′-thiocytidine, Thiarabine (T-araC), 2′-Deoxy-4′-thio-5-azacytidine or Apricitarabine (ATC).

More preferably, the APIs, intermediates or prodrugs thereof produced by the use of the enzymes disclosed herein, are selected from: Capecitabine, Decitabine (aza-dCyd or DAC), 5-Azacytidine (aza-Cyd), Cytarabine (ara-C), and still more preferably the described process is particularly intended to the industrial production of Capecitabine or Cytarabine and, respectively, intermediates or prodrugs thereof.

Also included in present invention are recombinant expression vectors comprising sequences encoding a nucleoside phosphorylase enzymatic activity (NPs) operably linked to one or more control sequences that direct the expression or overexpression of said nucleoside phosphorylases in a suitable host. In some aspects, a recombinant expression vector according to present invention is any carrying and expressing or overexpressing genes encoding NP enzymatic activities existing in thermophilic and hyperthermophilic Archaea, preferably Crenarchaeote and more preferably from the class Thermoprotei, such as: AI1RW90 (A1RW90THEPD), for the hypothetical protein from Thermofilum pendens (strain Hrk 5); Q97Y30 (Q97Y30SULSO), for the hypothetical protein from Sulfolobus solfataricus; A3DME1 (A3DME1 STAMF), for the hypothetical protein from Staphylothermus marinus (strain ATCC 43588/DSM 3639/FI); Q9YA34 (Q9YA34AERPE), for the hypothetical protein from Aeropyrum pernix; A2BJ06 (A2BJ06HYPBU) for the hypothetical protein from Hyperthermus butylicus (strain DSM 5456/JCM 9403); and D9PZN7 (D9PZN7ACIS3) for the hypothetical protein from Acidilobus saccharovorans (strain DSM 16705/VKM B-2471/345)

TABLE 3
Encoded NP enzymes

| Acces No. | | | | | |
|---|---|---|---|---|---|
| Uniprot | | | | | |
| Genbank | | | | | SEQ ID NO |
| (NCBI) | Entry name | Protein names | Organism | Gene names | (DNA/Protein) |
| A1RW90 | A1RW90_THEPD | Purine-nucleoside | Thermofilum pendens | Tpen_0060 | 5/6 |
| YP_919473.1. | | phosphorylase | (strain Hrk 5) | | |
| Q97Y30 | Q97Y30_SULSO | Purine nucleoside | Sulfolobus solfataricus | deoD SSO1519 | 1/3 |
| NP_342951.1 | | phosporylase | (strain ATCC 35092/ | | |
| | | (DeoD) | DSM 1617/JCM | | |
| | | | 11322/P2) | | |
| A3DME1 | A3DME1_STAMF | Purine-nucleoside | Staphylothermus | Smar_0697 | 7/8 |
| YP_001040709.1. | | phosphorylase | marinus (strain ATCC | | |
| | | | 43588/DSM 3639/ | | |
| | | | F1) | | |
| Q9YA34 | Q9YA34_AERPE | Uridine | Aeropyrum pernix | udp | 2/4 |
| NP_148386.2 | | phosphorylase | (strain ATCC 700893/ | APE_2105.1 | |
| | | | DSM 11879/JCM | | |
| | | | 9820/NBRC 100138/K1) | | |
| A2BJ06 | A2BJ06_HYPBU | Uridine | Hyperthermus | Hbut_0092 | 9/10 |
| YP_001012312.1 | | phosphorylase | butylicus (strain DSM | | |
| | | | 5456/JCM 9403) | | |
| D9PZN7 | D9PZN7_ACIS3 | Uridine | Acidilobus | ASAC_0117 | 11/12 |
| YP_003815556.1 | | phosphorylase | saccharovorans (strain | | |
| | | | DSM 16705/VKM | | |
| | | | B-2471/345-15) | | |

In some aspects, recombinant expression vectors according to present invention are carrying and expressing or overexpressing a nucleic acid sequence selected from: SEQ ID NO. 1, 2, 5, 7, 9 or 11; or
a) a nucleotide sequence which is the complement of SEQ ID. NO: 1, 2, 5, 7, 9 or 11; or
b) a nucleotide sequence which is degenerate with SEQ ID. NO: 1, 2, 5, 7, 9 or 11; or
c) a nucleotide sequence hybridizing under conditions of high stringency to SEQ ID. NO:1, 2, 5, 7, 9 or 11; to the complement of SEQ ID. NO:1, 2, 5, 7, 9 or 11; or to a hybridization probe derived from SEQ ID. NO: 1, 2, 5, 7, 9 or 11; or their complement thereof; or
d) a nucleotide sequence having at least 80% sequence identity with SEQ ID. NO: 1, 2, 5, 7, 9 or 11; or
e) a nucleotide sequence having at least 59% sequence identity with SEQ ID. NO: 1, 2, 5, 7, 9 or 11; or
f) a nucleotide sequence encoding for an amino acid sequence selected from: SEQ ID. NO: 3, 4, 6, 8, 10 or 12,

The invention also covers the use of the recombinant expression vectors previously mentioned, for the production either of recombinant NPs or for the production of active pharmaceutical ingredients (APIs), intermediates or prodrugs thereof, being those APIs or their intermediates, nucleoside analogues (NAs) particularly useful as anti-cancer or anti-viral medicaments. Particularly APIs, intermediates or prodrugs thereof produced by the aforesaid use are selected from: Capecitabine, Decitabine (aza-dCyd or DAC), 5-Azacytidine (aza-Cyd), Cytarabine (ara-C), Enocitabine (BH-AC), Gemcitabine (dFdC), Zalcitabine (ddC), Ibacitabine, Sapacitabine, 2′-C-cyano-2′-deoxy-1-β-D-arabino-pentofuranosylcytosine (CNDAC), Galocitabine, Valopricitabine (NM283), 2′-Deoxy-4′-thiocytidine, Thiarabine (T-araC), 2′-Deoxy-4′-thio-5-azacytidine and Apricitarabine (ATC).

In some aspects, the APIs, intermediates or prodrugs thereof produced are selected from: Capecitabine, Decitabine (aza-dCyd or DAC), 5-Azacytidine (aza-Cyd), Cytarabine (ara-C); and still more preferably the expression vectors described herein are particularly suitable for industrial production of Capecitabine or Cytarabine, intermediates or prodrugs thereof.

In some aspects, the previously mentioned use for the production of APIs, intermediates or prodrugs thereof is achieved by the production process and variants thereof, also previously detailed.

The invention also covers host cells comprising the recombinant expression vectors, previously described, particularly when said host cell is Escherichia coli. Included as part of the same inventive single linked concept, the use of host cells comprising the recombinant expression vectors as previously described, for the production of recombinant NPs (o PyNP), is also contemplated. Analogously, the use of host cells comprising the recombinant expression vectors as previously described, for the production of active pharmaceutical ingredients (APIs), intermediates or prodrugs thereof, being those APIs or their intermediates, nucleoside analogues (NAs) useful as anti-cancer or anti-viral medicaments, is also part of present invention. Particularly if those host cells comprising the recombinant expression vectors as previously described, are used for producing APIs, intermediates or prodrugs thereof selected from: Capecitabine, Decitabine (aza-dCyd or DAC), 5-Azacytidine (aza-Cyd), Cytarabine (ara-C), Enocitabine (BH-AC), Gemcitabine (dFdC), Zalcitabine (ddC), Ibacitabine, Sapacitabine, 2′-C-cyano-2′-deoxy-1-β-D-arabino-pentofuranosylcytosine (CNDAC), Galocitabine, Valopricitabine (NM283), 2′-Deoxy-4′-thiocytidine, Thiarabine (T-araC), 2′-Deoxy-4′-thio-5-azacytidine and Apricitarabine (ATC).

In some aspects, the APIs, intermediates or prodrugs thereof produced using the host cells of present invention, transformed with the recombinant expression vectors, previously described, are selected from: Capecitabine, Decitabine (aza-dCyd or DAC), 5-Azacytidine (aza-Cyd), Cytarabine (ara-C); and still more preferably the host transformed cells described herein are particularly suitable for industrial production of Capecitabine or Cytarabine, intermediates or prodrugs thereof.

In some aspects, the previously mentioned use is achieved by the production process and variants thereof, also previously detailed, of NAs as APIs, intermediates or prodrugs thereof.

All the references cited throughout the instant applicant, including scientific publications, patents, and patent application publications are herein incorporated by reference in their entireties. Aspects of the present disclosure can be further defined by reference to the following non-limiting examples, which describe in detail preparation of certain compounds of the present disclosure. It will be apparent to those skilled in the art that many modifications, both to materials and methods, can be practiced without departing from the scope of the present disclosure.

### EXAMPLES

### Example 1

### Synthesis of 5-Fluoro-5′-Deoxycytidine Departing from Unmodified 5-Fluorocytosine and Deoxyuridine

A solution of 2.5 mM 5-fluorocytosine and 8.0 mM 5′-deoxyuridine in 30 mM aqueous phosphate buffer at pH 7 and 10% DMSO was heated at 600° C. during 30 min. Then, PyNP (5.4 U/μmol_{base}, was added and the reaction was stirred at 60° C. during 4 hours under the same conditions. Then, the crude reaction was filtered through a 10 KDa membrane, and a portion was diluted and analyzed by HPLC. The expected product 5-Fluoro-5′-deoxycytidine was not detected by UV-DAD (ultraviolet-diode array detection).

### Example 2

### Synthesis of 5-Fluoro-5′-Deoxycytidine Departing from Unmodified 5-Fluorocytosine and Chloro-5′-Deoxyuridine

A solution of 2.5 mM 5-fluorocytosine and 8.6 mM 5-chloro-5′-deoxyuridine in 30 mM aqueous phosphate buffer at pH 7 and 10% DMSO was heated at 60° C. during 30 min. Then, PyNP (5.4 U/μmol_{base}) was added and the reaction was stirred at 60° C. during 4 hours under the same conditions. Then, the crude reaction was filtered through a 10 KDa membrane, and a portion was diluted and analyzed by HPLC. The expected product 5-Fluoro-5′-deoxycytidine was not detected by UV-DAD.

### Example 3

### Synthesis of Cytarabine Departing from Unmodified Cytosine and Arabinofuranosyluracil

A solution of 3.0 mM cytosine and 10 mM 9-(b-D-arabinofuranosyl)uracil in 30 mM aqueous phosphate buffer at pH 7 and 10% DMSO was heated at 60° C. during 30 min. Then, PyNP (4.4 U/μmol_{base}) was added and the reaction was stirred at 60° C. during 4 hours under the same conditions. Then, the crude reaction was filtered through a 10 KDa membrane, and a portion was diluted and analyzed by HPLC. The expected product 1-(b-D-arabinofuranosyl)cytosine (Cytarabine) was not detected by UV-DAD, but uracil from cytosine decomposition through deamination was obtained.

### Example 4

### Synthesis of Cytarabine Departing from Unmodified Cytosine and Arabinofuranosylcytosine

A solution of 3.0 mM cytosine and 7.6 mM 9-(b-D-arabinofuranosyl)adenine in 30 mM aqueous phosphate buffer at pH 7 and 10% DMSO was heated at 600° C. during 30 min. Then, PyNP (4.4 U/μmol_{base}) was added and the reaction was stirred at 60° C. during 4 hours under the same conditions. Then, the crude reaction was filtered through a 10 KDa membrane, and a portion was diluted and analyzed by HPLC. The expected product 1-(b-D-arabinofuranosyl)cytosine (Cytarabine) was not detected by UV-DAD, but hypoxanthine from adenine deamination and 9-(b-D-arabinofuranosyl)hypoxanthine from the substrate deamination were obtained.

### Example 5

### Synthesis of Cytarabine Departing from Unmodified Cytosine and Arabinofuranosylhypoxanthine

A solution of 3.0 mM cytosine and 7.5 mM 9-(b-D-arabinofuranosyl)hypoxanthine in 30 mM aqueous phosphate buffer at pH 7 and 10% DMSO was heated at 60° C. during 30 min. Then, PyNP (4.4 U/μmol_{base}) was added and the reaction was stirred at 60° C. during 4 hours under the same conditions. Then, the crude reaction was filtered through a 10 KDa membrane, and a portion was diluted and analyzed by HPLC. The expected product 1-(b-D-arabinofuranosyl)cytosine (Cytarabine) was not detected by UV-DAD.

### Example 6

### Synthesis of Cytarabine Departing from Unmodified Cytosine and Arabinofuranosylguanine

A solution of 3.0 mM cytosine and 8.6 mM 9-(b-D-arabinofuranosyl)guanine in 30 mM aqueous phosphate buffer at pH 7 and 10% DMSO was heated at 60° C. during 30 min. Then, PyNP (4.4 U/μmol_{base}) was added and the reaction was stirred at 60° C. during 4 hours under the same conditions. Then, the crude reaction was filtered through a 10 KDa membrane, and a portion was diluted and analyzed by HPLC. The expected product 1-(b-D-arabinofuranosyl)cytosine (Cytarabine) was not detected.

### Example 7

### Synthesis of pentyl (5-fluoro-2-oxo-1,2-dihydropyrimidin-4-yl)carbamate

Under inert atmosphere, pentyl chloroformate (5 g, 1.0 equiv) was added to a stirred solution of 5-fluorocytosine (1.0 equiv) in anhydrous pyridine, and the reaction was heated to 60° C. After 2 hours, as no more conversion was observed, the reaction was quenched by cooling at room temperature. After that, the crude reaction was extracted using AcOEt and HCl 1N, and the organic phase was dried over Na₂SO₄ and the solvent was evaporated. The white solid obtained was chromatographically purified using SiO₂ and AcOEt as mobile phase, yielding the desired product (88%).

### Example 8

### Synthesis of N-(2-oxo-1,2-dihydropyrimidin-4-yl)acetamide

Under inert atmosphere, triethylamine (18.1 μL, 0.13 mmol) and acetyl chloride (9.2 μL, 0.13 mmol) were added to a stirred solution of cytosine (10.0 mg, 0.09 mmol) in anhydrous DMF at room temperature during 25 hours. After that, the solvent was evaporated, and the yellowish solid obtained was washed with water, filtered and dried in vacuo, rendering the expected product in 55% yield.

### Example 9

### Synthesis of pentyl (2-oxo-1,2-dihydropyrimidin-4-yl)carbamate

Under inert atmosphere, pentyl chloroformate (0.8 mmol, 1.0 equiv) was added dropwise to a stirred solution of cytosine (0.8 mmol, 1.0 equiv) in anhydrous pyridine, and the reaction was heated to 60° C. After 2 hours, as no more conversion was observed, the reaction was quenched by cooling at room temperature. After that, the crude reaction was extracted using AcOEt and HCl 1N, and the organic phase was dried over Na₂SO₄ and the solvent was evaporated. The white solid obtained was chromatographically purified using SiO₂ and AcOEt as mobile phase, yielding the desired product.

### Example 10

### Synthesis of N⁴-pentyloxycarbonyl-5′-deoxy-5-fluorocytidine (Capecitabine) Departing from Uridine

A solution of 2.7 mM pentyl (5-fluoro-2-oxo-1,2-dihydropyrimidin-4-yl)carbamate and 8.0 mM 5′-deoxyuridine in 30 mM aqueous phosphate buffer at pH 7 and 10% DMSO was heated at 60° C. during 30 min. Then, PyNP (5.0 U/μmol_{base}) was added and the reaction was stirred at 60° C. during 4 hours under the same conditions. Then, the crude reaction was filtered through a 10 KDa membrane, and a portion was diluted and analyzed by HPLC. The expected product N⁴-pentyloxycarbonyl-5′-deoxy-5-fluorocytidine (Capecitabine) was detected with a yield of 68%, in comparison to Reference Example 1, where the final product was not formed or detected.

### Example 11

### Synthesis of N⁴-pentyloxycarbonyl-5′-deoxy-5-fluorocytidine (Capecitabine) Departing from Deoxyuridine

A solution of 2.5 mM pentyl (5-fluoro-2-oxo-1,2-dihydropyrimidin-4-yl)carbamate and 8.6 mM 5′-deoxyuridine in 30 mM aqueous phosphate buffer at pH 7 and 10% DMSO was heated at 60° C. during 30 min. Then, PyNP (5.4 U/μmol_{base}) was added and the reaction was stirred at 60° C. during 4 hours under the same conditions. Then, the crude reaction was filtered through a 10 KDa membrane, and a portion was diluted and analyzed by HPLC. The expected product N⁴-pentyloxycarbonyl-5′-deoxy-5-fluorocytidine (Capecitabine) was detected with a yield of 77%, in comparison to Reference Example 2, where the final product was not formed or detected.

### Example 12

### Synthesis of 9-(b-D-Arabinofuranosyl)Uracil (Cytarabine) Departing from Arabinofuranosyluracil

A solution of 2.5 mM pentyl (2-oxo-1,2-dihydropyrimidin-4-yl)carbamate and 8.6 mM 9-(b-D-arabinofuranosyl)uracil in 30 mM aqueous phosphate buffer at pH 7 and 10%/o DMSO was heated at 60° C. during 30 min. Then, PyNP (5.4 U/μmol_{base}) was added and the reaction was stirred at 60° C. during 4 hours under the same conditions. Then, the crude reaction was filtered through a 10 KDa membrane, and a portion was diluted and analyzed by HPLC. The expected product N⁴-pentyloxycarbonylcytidine was detected.

N⁴-position can be optionally deprotected to furnish 9-(b-D-arabinofuranosyl)uracil (Cytarabine).

### Example 13

### Synthesis of 9-(b-D-Arabinofuranosyl)Uracil (Cytarabine) Departing from Arabinofuranosyladenine

A solution of 2.5 mM pentyl (2-oxo-1,2-dihydropyrimidin-4-yl)carbamate and 8.6 mM 9-(b-D-arabinofuranosyl)adenine in 30 mM aqueous phosphate buffer at pH 7 and 10% DMSO was heated at 60° C. during 30 min. Then, PyNP (5.4 U/μmol_{base}) was added and the reaction was stirred at 60° C. during 4 hours under the same conditions. Then, the crude reaction was filtered through a 10 KDa membrane, and a portion was diluted and analyzed by HPLC. The expected product N⁴-pentyloxycarbonylcytidine was detected.

N⁴-position can be optionally deprotected to furnish 9-(b-D-arabinofuranosyl)uracil (Cytarabine).

1. A method for producing a cytosine nucleoside analogue, intermediate or prodrug thereof of formula I 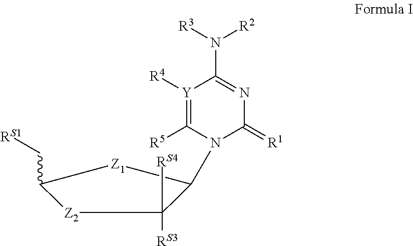
by chemo-enzymatic or enzymatic synthesis, wherein,
Z₁ is O, CH₂, S, NH;
Z₂ is independently selected from Z₁: O, C(R^{S2}R^{S5}), S(R^{S2}R^{S5}), S(R^{S2}), S(R^{S5}), preferably, a group SO or SO₂; N(R^{S2}R^{S5}), N(R^{S2}), N(R^{S5});
R^{S1} is hydrogen, OH, ether or ester thereof selected from: 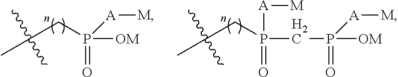
is n is 0 or 1, A is oxygen or nitrogen, and each M is independently hydrogen, an optionally substituted alkyl chain, an optionally substituted alkenyl chain, an optionally substituted alkynyl chain, an optionally substituted aryl optionally linked to P by an optionally substituted alkyl, alkenyl or alkynyl chain, an optionally substituted heterocycle optionally linked to P by an optionally substituted alkyl, alkenyl or alkynyl chain, or a pharmaceutically acceptable counter-ion such as, but not restricted to, sodium, potassium, ammonium or alkylammonium;
R^{S2} is hydrogen, OH or an ether or ester residue thereof, halogen, CN, NH₂, SH, C≡CH, N₃;
R^{S3} is hydrogen, in case of NA derived from 2′-deoxyribonucleosides or arabinonucleosides or being selected from: OH, NH₂, halogen, OCH₃, when the NA is derived from ribonucleosides;
R^{S4} is hydrogen, OH or an ether or ester residue thereof, NH₂, halogen, CN;
providing R^{S1} and R^{S4} are different when both were ethers or esters of OH residues;
R^{S5} is hydrogen, OH or an ether or ester residue thereof, NH₂ or halogen, in the case that Z₂ is different from oxygen;
R¹ is O, CH₂, S, NH;
R² is hydrogen, an optionally substituted C₄₋₄₀ alkyl chain, an optionally substituted alkenyl chain, an optionally substituted alkynyl chain, an optionally substituted aryl linked to N by an optionally substituted alkyl, alkenyl or alkynyl chain, an optionally substituted heterocycle linked to N by an optionally substituted alkyl, alkenyl or alkynyl chain, COR⁶, CONR⁶R⁷, CO₂R⁶, C(S)OR⁷, CN, SR⁶, SO₂R⁶, SO₂R⁶R⁷, CN, P(O)aryl, P(O)heterocycle, P(S)aryl, P(S)heterocycle, P(O)O₂R⁸;
R³ is hydrogen, an optionally substituted C₄, alkyl chain, an optionally substituted alkenyl chain, an optionally substituted alkynyl chain, an optionally substituted aryl linked to N by an optionally substituted alkyl, alkenyl or alkynyl chain, an optionally substituted heterocycle linked to N by an optionally substituted alkyl, alkenyl or alkynyl chain, COR⁶, CONR⁶R⁷, CO₂R⁶, C(S)OR⁷, CN, SR⁶, SO₂R⁶, SO₂R⁶R⁷, CN, P(O)aryl, P(O)heterocycle, P(S)aryl, P(S)heterocycle, P(O)O₂R⁸;
being R³ and R² independent one from each other; and providing that at least one R₂ or R₃ is different from hydrogen;
R₄ is hydrogen, OH, NH₂, SH, halogen; optionally substituted alkyl chain; optionally substituted alkenyl chain; optionally substituted alkynyl chain, trihaloalkyl, OR⁶, NR⁶R⁷, CN, COR⁶, CONR⁶R⁷, CO₂R⁶, C(S)OR⁶, OCONR⁶R⁷, OCO₂R⁶, OC(S)OR⁶, NHCONR⁶R⁷, NHCO₂R⁶, NHC(S)OR⁶, SO₂NR⁶R⁷, an optionally substituted aryl linked to Y by an optionally substituted alkyl, alkenyl or alkynyl chain, an optionally substituted heterocycle linked to Y by an optionally substituted alkyl, alkenyl or alkynyl chain, and any optionally substituted heterocycle or optionally substituted aryl of, independently, R², R³, R⁴ or R⁵, selected from: 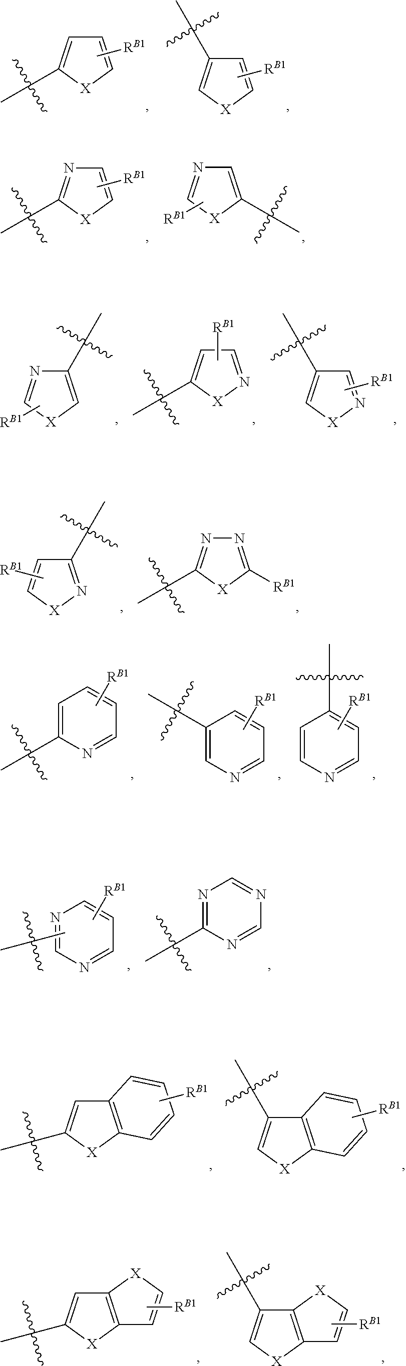 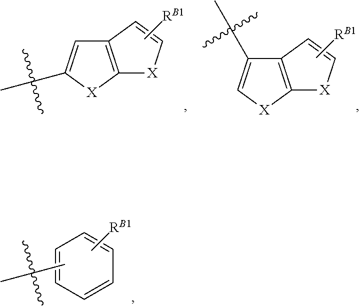
providing Y is a carbon or sulphur atom and, alternatively, R⁴ being absent, providing Y is a nitrogen atom;
wherein
X is O, S, N—R^{B2}, Se; R^{B1} is H, OH, NH₂, SH, straight or branched C₁₋₁₀ alkyl, F, Cl, Br, I, X—R^{B2}, —C≡C—R^{B2}, CO₂R^{B2}; R^{B2} is H, OH, NH₂, straight or branched C₁₋₅ alkyl, phenyl;
R⁵ is hydrogen, OH, NH₂, SH, halogen, an optionally substituted alkyl chain, an optionally substituted alkenyl chain, an optionally substituted alkynyl chain, trihaloalkyl, OR⁶, NR⁶R⁷, CN, COR⁶, CONR⁶R⁷, CO₂R⁶, C(S)OR⁶, OCONR⁶R⁷, OCO₂R⁶, OC(S)OR⁶, NHCONR⁶R⁷, NHCO₂R⁶, NHC(S)OR⁶, SO₂NR⁶R⁷; CH₂-heterocyclic ring, CN;
and any optionally substituted heterocycle or optionally substituted aryl of, independently, R², R¹, R⁴ or R⁵, selected from: 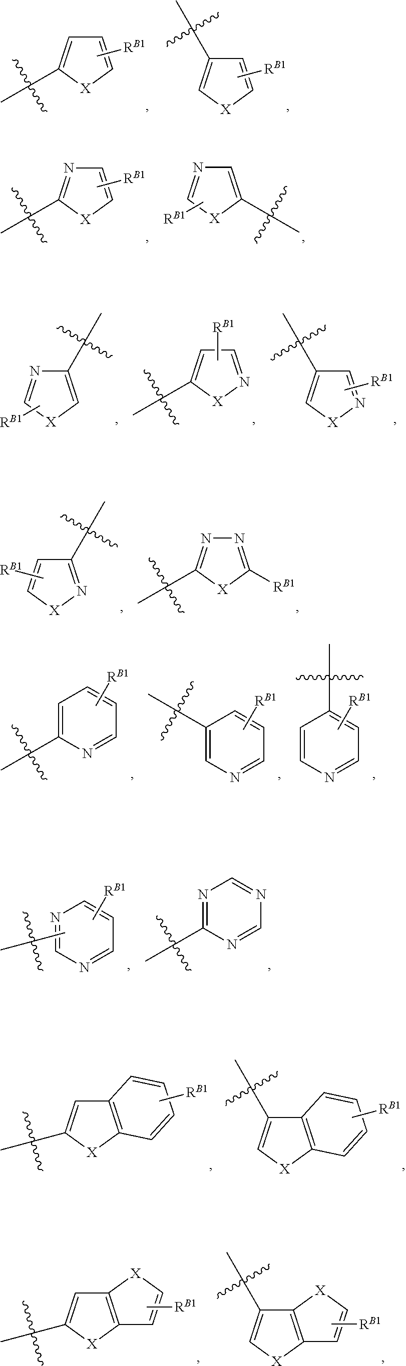 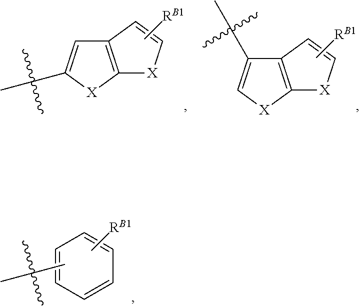
wherein
X is O, S, N—R^{B2}, Se; R^{B1} is H, OH, NH₂, SH, straight or branched C₁₋₁₀ alkyl, F, Cl, Br, I, X—R^{B2}, —C≡C—R^{B2}, CO₂R^{B2}; R^{B2} is H, OH, NH₂, straight or branched C₁₋₅ alkyl, phenyl;
R⁶ and R⁷ are independently of each other hydrogen, optionally substituted alkyl chain, optionally substituted alkenyl chain, optionally substituted alkynyl chain, heterocyclic or optionally substituted aryl;
R⁸ is hydrogen, an optionally substituted alkyl chain, an optionally substituted alkenyl chain, an optionally substituted alkynyl chain, an optionally substituted aryl an optionally substituted heterocycle;
Y is C, N, S;
wherein the method comprises:
(i) chemically reacting a precursor of the cytosinic nucleobase of formula II, wherein Y, R¹, R⁴, R⁵, R⁶ and R⁷, are defined as above, with suitable reagents for modifying its amino group at N⁴ position in order to incorporate the proper substitution described as substituent R² and R³, said modified cytosinic nucleobase of formula II formed thereof, being optionally purified by conventional purification methods; or alternatively, the process departs directly from the starting products represented by a cytosinic nucleobase of formula II as such. 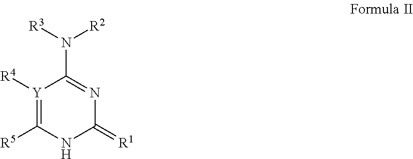
(ii) biocatalytically reacting the above mentioned modified cytosinic nucleobase of formula II, with a suitable nucleoside analog substrate of formula III, 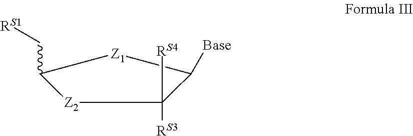
wherein Z₁, Z₂, R^{S1}, R^{S2}, R^{S3}, R^{S4}, R^{S5} are defined as above and the Base is selected from: uracil, adenine, cytosine, guanine, thymine, hypoxanthine, xanthine, thiouracil, thioguanine, 9-H-purine-2-amine, 7-methylguanine, 5-fluorouracil, 5-bromouracil, 5-chlorouracil, 5,6-dihydrouracil, 5-methylcytosine and 5-hydroxymethylcytosine, pteridone, and any substituted derivative thereof;
wherein the aforesaid reaction carried out in step ii), comprises the addition, in a suitable reaction aqueous medium and under suitable reaction conditions, of a nucleoside phosphorylase enzyme, either a pyrimidine nucleoside phosphorylase enzyme, a purine nucleoside phosphorylase enzyme or combinations thereof, to a mixture of starting materials comprising a cytosine nucleobase of formula II and a nucleoside analogue of formula III,
(iii) optionally, deprotecting the amino group at N⁴ position in cytosinic nucleoside analogue in order to recover the free primary amino N⁴ at the cytosinic nucleoside analogue of formula I which was further purified by conventional purification methods.

2. The method according to claim 1, wherein the cytosinic nucleobase of formula II to be transferred by the pyrimidine nucleoside phosphorylase enzyme is selected from:

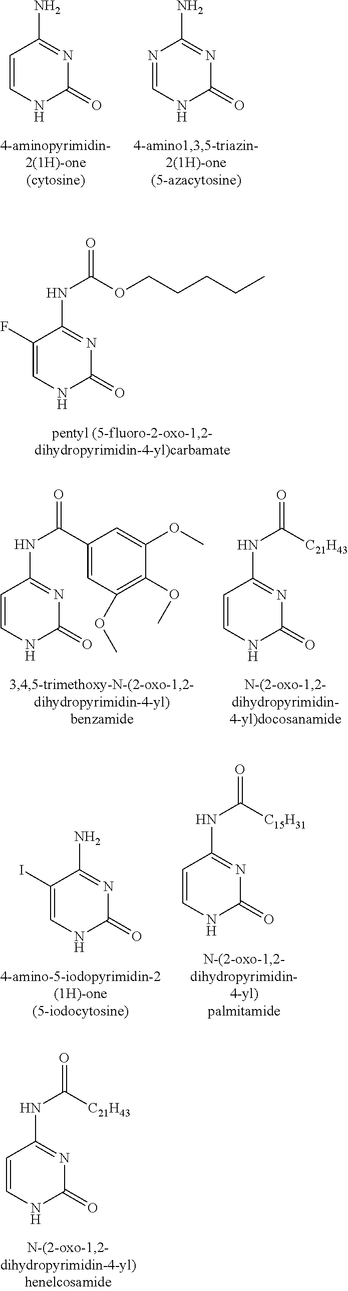

3. The method according to claim 1, wherein the nucleoside analog, intermediate or prodrug thereof produced is selected from: Capecitabine, Decitabine, 5-Azacytidine, Cytarabine, Enocitabine, Gemcitabine, Zalcitabine, Ibacitabine, Sapacitabine, 2′-C-cyano-2′-deoxy-1-β-D-arabino-pentofuranosylcytosine, Galocitabine, Valopricitabine, 2′-Deoxy-4′-thiocytidine, Thiarabine, 2′-Deoxy-4′-thio-5-azacytidine and Apricitarabine.

4. The method according to claim 1, wherein the source of native, mutants or variants thereof, or of recombinant nucleoside phosphorylase enzyme, is mesophilic, thermophilic or hyperthermophilic organisms.

5. The method according to claim 1, wherein the source of native, mutants or variants thereof, or of recombinant nucleoside phosphorylase enzyme is selected from Archaea or bacteria.

6. The method according to claim 5, wherein the nucleoside phosphorylase enzyme is isolated from an Archaea selected from: Sulfolobus solfataricus or Aeropyrum pernix.

7. The method according to claim 1, wherein the nucleoside phosphorylase enzyme, or a functional part thereof, is encoded by a nucleotide sequence selected from: SEQ ID NO. 1, 2, 5, 7, 9 or 11; or
a) a nucleotide sequence which is the complement of SEQ ID. NO:1, 2, 5, 7, 9 or 11; or
b) a nucleotide sequence which is degenerate with SEQ ID. NO: 1, 2, 5, 7, 9 or 11; or
c) a nucleotide sequence hybridizing under conditions of high stringency to SEQ ID. NO: 1, 2, 5, 7, 9 or 11; to the complement of SEQ ID. NO:1, 2, 5, 7, 9 or 11; or to a hybridization probe derived from SEQ ID. NO: 1, 2, 5, 7, 9 or 11; or their complement thereof; or
d) a nucleotide sequence having at least 80% sequence identity with SEQ ID. NO:1, 2, 5, 7, 9 or 11; or
e) a nucleotide sequence having at least 59% sequence identity with SEQ ID. NO: 1, 2, 5, 7, 9 or 11; or
f) a nucleotide sequence encoding for an amino acid sequence selected from: SEQ ID. NO: 3, 4, 6, 8, 10 or 12.

8. The method according to claim 3, wherein the nucleoside analogue produced is Capecitabine; the ribonucleoside used as starting material is selected from: 5′-deoxyuridine, 5′-deoxy-5-methyluridine or 5′-deoxy-5-chlorouridine; and the nucleobase used as starting material to be transferred by the Pyrimidine Nucleoside Phosphorilase enzyme is pentyl (5-fluoro-2-oxo-1,2-dihydropyrimidin-4-yl)carbamate.

9. The method according to claim 3, wherein the nucleoside analogue produced is Cytarabine; the ribonucleoside used as starting material is 9-(b-D-arabinofuranosyl)uracil; and the nucleobase used as starting material to be transferred by the Pyrimidine Nucleoside Phosphorilase enzyme is N-(2-oxo-1,2-dihydropyrimidin-4-yl) pentanamide.

10. A method to produce a cytosinic nucleoside analogue, intermediate or prodrug thereof for the treatment of cancer or viral infection in a subject in need thereof comprising the use of
a. a native mesophilic, thermophilic or hyperthermophilic nucleoside phosphorylase, or a combination thereof;
b. a mutant mesophilic, thermophilic or hyperthermophilic nucleoside phosphorylase, or a combination thereof;
c. a variant mesophilic, thermophilic or hyperthermophilic nucleoside phosphorylase, or a combination thereof;
d. a recombinant mesophilic, thermophilic or hyperthermophilic nucleoside phosphorylase, or a combination thereof;
e. a functional fragment of a mesophilic, thermophilic or hyperthermophilic nucleoside phosphorylase, or a combination thereof;
f. a recombinant expression vector comprising a sequence encoding a nucleoside phosphorylase according to (a), (b), (c), (d) or (e), operably linked to one or more control sequences that direct the expression or overexpression of said nucleoside phosphorylase in a suitable host; or
g. a microorganism or a host cell containing (a), (b), (c), (d), (e) or (f) or a combination thereof.

11. The method according to claim 10, wherein the mesophilic, thermophilic or hyperthermophilic nucleoside phosphorylase enzyme, or a functional part thereof, is encoded by a nucleotide sequence selected from: SEQ ID NO. 1, 2, 5, 7, 9 or 11; or
a) a nucleotide sequence which is the complement of SEQ ID. NO:1, 2, 5, 7, 9 or 11; or
b) a nucleotide sequence which is degenerate with SEQ ID. NO: 1, 2, 5, 7, 9 or 11; or
c) a nucleotide sequence hybridizing under conditions of high stringency to SEQ ID. NO: 1, 2, 5, 7, 9 or 11; to the complement of SEQ ID. NO:1, 2, 5, 7, 9 or 11; or to a hybridization probe derived from SEQ ID. NO: 1, 2, 5, 7, 9 or 11; or their complement thereof; or
d) a nucleotide sequence having at least 80% sequence identity with SEQ ID. NO:1, 2, 5, 7, 9 or 11; or
e) a nucleotide sequence having at least 59% sequence identity with SEQ ID. NO:1, 2, 5, 7, 9 or 11; or
f) a nucleotide sequence encoding for an amino acid sequence selected from: SEQ ID. NO: 3, 4, 6, 8, 10 or 12.

12. The method according to claim 10, wherein the cytosinic nucleoside analogue, intermediate or prodrug thereof produced is selected from: Capecitabine, Decitabine, 5-Azacytidine, Cytarabine, Enocitabine, Gemcitabine, Zalcitabine, Ibacitabine, Sapacitabine, 2′-C-cyano-2′-deoxy-1-β-D-arabino-pentofuranosylcytosine, Galocitabine, Valopricitabine, 2′-Deoxy-4′-thiocytidine, Thiarabine, 2′-Deoxy-4′-thio-5-azacytidine and Apricitarabine.

13. The method according to claim 12, wherein the cytosinic nucleoside analogue, intermediate or prodrug thereof produced is Capecitabine.

14. The method according to claim 12, wherein the cytosinic nucleoside analogue, intermediate or prodrug thereof produced is Cytarabine.


SEQUENCE LISTING (12 sequences)

SEQ ID NO: 1 - DNA, 825 bp, Sulfolobus solfataricus
  source 1..825: /mol_type=“unassigned DNA” /note=“P2 Purine nucleoside phosporylase (deoD)” /organism=“Sulfolobus solfataricus”
gtgccatttttagaaaatggttccatggtatatggtgatttcattagaaatcaagaggtaagaaaaagaattacaaaggaagaacttgggatagaagaagacgaaatcccggaaagggtagttgtaacacctatgccatttaatactcaatttcctaaaaactttgaagatactttaactaacttaggaattaaagtaaataggttaaaagtggaagaccaaatacttagacaattcggaggaaatttattgcttgaaaaagacggtaatagaggatttattgcgttcataggcagaggtctgatagatttcactgagaggataaggattttagctacagtttcgcgcattaaagatatattatttattggtactgcaggatcgttatctaatgaaatattaataggagatctaaatataccaaaatacgccatcccattcgaaaacgtaagtgatttttacgctgatcctaccatagcaattccacaagctgatgaaaagttgctgaacgaagtttatgagtacgctgaggaaactggagttaaaacccactcaaccttacatgcaacactacttttcccttattccgaaactactgagttcctaaactacttattaaatatcggcgtttctacgatagatatggaagtcagtgctttttataagatgtctagattttacggtaaaagagctgttgcagtattacgaatttcagatatgcctttaatagaactgcataagcaagaggaattgattaaggcaagaagggaaattgcagttaatgctgttttcagaattaccttaagattcttaaaactgatttaa

SEQ ID NO: 2 - DNA, 849 bp, Aeropyrum pernix K1
  source 1..849: /mol_type=“unassigned DNA” /note=“udp uridine phosphorylase” /organism=“Aeropyrum pernix K1”
ttgggagacgagagtctaaggagcgccgcccgtcccgagggggagggagggctgcagtaccatctgagggtcaggaggggggatgtggcccgctacgttctcctcccgggagaccccgagaggacagaccttatagcccgcctctgggatgaagcgaggcttgtagcgcaccaccgggagtacaggacgtggaccggcttctacaaggggacatcgataagtgtaacaagcaccgggataggctctcccagcacggcgatagccgttgaggagctgctgagggttggagccgagactttcataagagtaggcactatgggcggtataagggaggatctgcggcccggcaccctggttatagggagtgcggcggttaggatggaggggacgagcggccagtacgctccccgggggttcccagcggccgccagctatgacgttgtggcggcgctggtggaggctgctgaggcgctcggggttaggtatgaggttggcgttgttgccagcacggacagcttctacctgggccaggggaggccggggtacggggggtatatgacgccggaggcttcggaagtcatacccctcctcaggtcagccggcgtcctcggcttcgagatggaggcctccgccctcttcaccctatcccagctctacggcgccagggcagggtgcgtgtgcgcggtagtggcaaacagggttagcggggagtttgtggtaaacgcgggggttgaagacgctgctagggttgcctccgaggcggtagccatactagcaggctgggacagggagagggagaagaggggtaagaaatggttttacccgagcctggcgtgcagacgcacatag

SEQ ID NO: 3 - protein, 274 aa, Sulfolobus solfataricus P2
  Purine nucleoside phosporylase (DeoD)
MPFLENGSMVYGDFIRNQEVRKRITKEELGIEEDEIPERVVVTPMPFNTQFPKNFEDTLTNLGIKVNRLKVEDQILRQFGGNLLLEKDGNRGFIAFIGRGLIDFTERIRILATVSRIKDILFIGTAGSLSNEILIGDLNIPKYAIPFENVSDFYADPTIAIPQADEKLLNEVYEYAEETGVKTHSTLHATLLFPYSETTEFLNYLLNIGVSTIDMEVSAFYKMSRFYGKRAVAVLRISDMPLIELHKQEELIKARREIAVNAVFRITLRFLKLI

SEQ ID NO: 4 - protein, 282 aa, Aeropyrum pernix K1 [sequence omitted]

SEQ ID NO: 5 - DNA, 720 bp, Thermofilum pendens Hrk 5
  source 1..720: /mol_type=“unassigned DNA” /note=“purine-nucleoside phosphorylase” /organism=“Thermofilum pendens Hrk 5”
gtggctaagccgttacacatactcgcaaagccggaggacatagcccccagggttatcgcctcgggggaccccgccagagtgaagcaactctcaagctacctcgataaccccaggctggtgaacgagaacaggggcttcctggtatacaccggcacgtacaagggggtacccgtgactgttgctacccacatgataggtgctccctccgccgcgatagtcttcgaggagctcataatgctgggtgcgaagctgatagtcaggttcggcacctgcggcggcttcctgccggagatgcgcgtaggggacttcgtgatagcgacgggcgcctcgtacagcggtgggggaacaatgaatacctacagccccggcgagtgcatggccgccgtgccggactacgacgtgctcagcgcactcgtcgagagcgcttcgaggcacgggttgaagtacttcctggggcccgtggttagcagcgataacttctactcaggtatagagtacctgaacaggtggataaacaggggcatgatagccgtcgacatggaggctgccacgctgttcgtcgtcggcaggcttaggcgcgtgaagaccggtgcctccttcgtcgtgagcgacgtgatcggtgaggcttacaagaagatggcgacggccgaggagctacgcgaggctgtcgacaaggcttcgagagccgtgctagacgcggttataagcgtgaaagtttga

SEQ ID NO: 6 - protein, 239 aa, Thermofilum pendens Hrk 5
  Purine-nucleoside phosphorylase
MAKPLHILAKPEDIAPRVIASGDPARVKQLSSYLDNPRLVNENRGFLVYTGTYKGVPVTVATHMIGAPSAAIVFEELIMLGAKLIVRFGTCGGFLPEMRVGDFVIATGASYSGGGTMNTYSPGECMAAVPDYDVLSALVESASRHGLKYFLGPVVSSDNFYSGIEYLNRWINRGMIAVDMEAATLFVVGRLRRVKTGASFVVSDVIGEAYKKMATAEELREAVDKASRAVLDAVISVKV

SEQ ID NO: 7 - DNA, 717 bp, Staphylothermus marinus F1
  source 1..717: /mol_type=“unassigned DNA” /note=“Purine-nucleoside phosphorylase ” /organism=“Staphylothermus marinus F1”
atgaagcctgctcttctaaaagatgttgcaggggttagtgatctagttattgtaatgggtgatcctgatagagtatacttattatcaacattgctggaaaaccccaagatcatatatgataggagaggaatagttgttgttaatggagaatataagggtagaaaaataacacttgccagccacggtattggttgtccaatggcatcaattatattggaggaactgggtatgcttggtgctaaaacaattattagaataggcacagctggctcacttgttgaaaacattggtttaggcgatattgtgttagtagcgggggctgggtacatgcttaatgggtgtggaaacaatatgtattctcccgaaataaatggaggaacaagcccagatcctctactgctcagcgaaatctatcattatttatcacattataatattaagccacatattggactagtatttacaagcgatgcattctatgcggaagaaaatataattgataaactaaccaataaaggattcatcgctgttgatatggagacagcaattctatacatgcttggatggatgaggaagtggaggacactatcaatactggtagtaagcaacagcctcgtgaagaaaacaccgttgcttacaacatatgagctcgcagagaagtttgttgaactagcaaaactagtattagaatatcttgcaagaaacactcagcactaa

SEQ ID NO: 8 - protein, 238 aa, Staphylothermus marinus F1
  Purine-nucleoside phosphorylase
MKPALLKDVAGVSDLVIVMGDPDRVYLLSTLLENPKIIYDRRGIVVVNGEYKGRKITLASHGIGCPMASIILEELGMLGAKTIIRIGTAGSLVENIGLGDIVLVAGAGYMLNGCGNNMYSPEINGGTSPDPLLLSEIYHYLSHYNIKPHIGLVFTSDAFYAEENIIDKLTNKGFIAVDMETAILYMLGWMRKWRTLSILVVSNSLVKKTPLLTTYELAEKFVELAKLVLEYLARNTQH

SEQ ID NO: 9 - DNA, 843 bp, Hyperthermus butylicus DSM 5456
  source 1..843: /mol_type=“unassigned DNA” /note=“Uridine phosphorylase” /organism=“Hyperthermus butylicus DSM 5456”
gtggagaggccctcggcgaaagcgccaaccgttgagggtaaaatgtaccatatcatgctcggcccaggcgaaattccgccctacgtcctcctgccaggcgatccaggtaggatagacgatatagtcgcgacgtgggatgaatggcgggagctagcatttcaccgcgagtatcgtagtgtcaagggcaggtataagggtgtggaaataggcgctgtcagcactggcataggcggaccgtcaactgcaattgccgtcgaggagctggctaggataggggtacatacgttcatccgcgtgggcaccactggcgctatacaaccggatatagaacttggaacagtcatcataggctatgcagctgtaagatatgatggtgctagtggtgagtatgctccgccagagtaccccgcagctgcgacacccgaggtggttctagctcttgttgaggctgctgagaggctcggggtaccataccgtgtcggcgtggtggcctcaacagcaacattccacttggggcagagccgcccagggttccgtgggtacgagtggagccgtagcagggagaggctagcagacctacagcgcatgggtgtcctcagcttcgagatggaggcggcgacgatattcactctcgcgagtctctatgggctgcgggcaggctgtgtctgcgccgcaatagccaaccgcgtgaccgacgagtttaaaccgggggtcggggttagggaggcgatactagtggctaatgaggctgtgaggatactagccgaggccgacagggaaaagggcaggaagccagctagtataacaacgctctacaacgccgtcagaaagctgtacggctag

SEQ ID NO: 10 - protein, 280 aa, Hyperthermus butylicus DSM 5456
  Uridine phosphorylase
MERPSAKAPTVEGKMYHIMLGPGEIPPYVLLPGDPGRIDDIVATWDEWRELAFHREYRSVKGRYKGVEIGAVSTGIGGPSTAIAVEELARIGVHTFIRVGTTGAIQPDIELGTVIIGYAAVRYDGASGEYAPPEYPAAATPEVVLALVEAAERLGVPYRVGVVASTATFHLGQSRPGFRGYEWSRSRERLADLQRMGVLSFEMEAATIFTLASLYGLRAGCVCAAIANRVTDEFKPGVGVREAILVANEAVRILAEADREKGRKPASITTLYNAVRKLYG

SEQ ID NO: 11 - DNA, 846 bp, Acidilobus saccharovorans 345-15
  source 1..846: /mol_type=“unassigned DNA” /note=“Uridine phosphorylase” /organism=“Acidilobus saccharovorans 345-15”
gtggaggagcgcatgtcttcagcttcaaggcccttcgactcggagggcagggcctaccacctgggcctcaggaagggcgacgtgccaaagtacgtgcttatgccaggcgaggttgagagggcctcaaggatagcctcctcctgggacaggagctggaggctggcccagaggagggagtactcaagcttcaggggcgtttacaggggcgttgacgtggcggtggtctccacgggcatagggggacctgccaccgccatagcagttgaggagctgctggagctcggcgctgacacgctgataagggttggaagcaccggcgccatacaggatgacatagaagttggggatataataataaccaccgccgcggtcaggatggacggcacgagctaccagtacgccccggccggctacccggcctcggccagctacgaggtaatcatggccctggtcgaggccgccgagagcctcggggtgaggtaccaccttggcatcacggcctctactgacagcttctacgtgggccaggggaggccgggctacgggggttacatgccgagctggtcaaggaacctcgtgcctgacctgaggcagatgagggtgctgaactttgagatggagagcgccaccctccttaccttagcaaatatatacggcttcagggcgggggcagtgcacgccgtctacgcccagagggttaaggatgagttcgttgcccacgctggcgaggagaacctgataaaggtggctgacgaggccgtgaggatacttcacgagtgggacgaggttaaggggagggccgggaagaggtacttctacccctcgctcctgaggcctggcccctga

SEQ ID NO: 12 - protein, 281 aa, Acidilobus saccharovorans 345-15
  Uridine phosphorylase
MEERMSSASRPFDSEGRAYHLGLRKGDVPKYVLMPGEVERASRIASSWDRSWRLAQRREYSSFRGVYRGVDVAVVSTGIGGPATAIAVEELLELGADTLIRVGSTGAIQDDIEVGDIIITTAAVRMDGTSYQYAPAGYPASASYEVIMALVEAAESLGVRYHLGITASTDSFYVGQGRPGYGGYMPSWSRNLVPDLRQMRVLNFEMESATLLTLANIYGFRAGAVHAVYAQRVKDEFVAHAGEENLIKVADEAVRILHEWDEVKGRAGKRYFYPSLLRPGP